(19) United States
(12) Patent Application Publication
(10) Pub. No.: US 2006/0052594 A1
(43) Pub. Date: Mar. 9, 2006

(54) New corticosteroids

(76) Inventors: Piero Del Soldato, Milano (IT); Ennio Ongini, Segrate (IT)

Correspondence Address: ARENT FOX PLLC, 1050 CONNECTICUT AVENUE, N.W., SUITE 400, WASHINGTON, DC, 20036, US

(21) Appl. No.: 10/501,335

(22) PCT Filed: Jan. 16, 2003
(86) PCT No.: PCT/EP03/00394

§ 371 Date: May 20, 2005

(30) Foreign Application Priority Data
Jan. 29, 2002 (IT) MI2002A000148

(51) Int. Cl. C07J 71/00

(52) U.S. Cl. 540/51

(57) ABSTRACT

Nitrooxy derivatives of steroidal compounds of general formula
B—X₁—NO₂ (I) or esters or salts thereof, wherein:
B is a steroidal radical, X₁ is a bivalent linking group comprising an aromatic or heterocyclic ring.

The present invention relates to steroidal compounds having an improved pharmacological activity and lower side effects and an improved receptor affinity on the specific receptors of endogenous steroids.

In particular the invention relates to steroidal compounds having an improved receptor affinity on the specific receptors of the endogenous steroids and having an improved pharmacological activity and lower side effects, in particular:
those affecting the bony tissue, such for example osteoporosis, osteonecrosis and myopathies, which in patients affected by asthma or by COPD (Chronic Obstructive Pulmunary Disease) can determine a remarkable reduction of the respiratory activity;
those affecting the gastrointestinal apparatus.

The invention relates to compounds having a steroidal structure in particular having not only an improved antiinflammatory activity at peripheral level, but also an improved anti-neurodegenerative activity, an improved antiarthritic activity, an improved immunodepressive activity, an improved angiostatic/angiogenetic and antiasthmatic activity; or usable in substitutive hormonal therapies, for example in the post-menopause therapy.

More specifically the present invention relates also to steroid compounds of the glucocorticoid class which can be used as bronchodilators in respiratory pathologies characterized by broncho-constrictive events.

The compounds according to the present invention are therapeutically useful in the treatment of illnesses wherein steroidal products are generally applied, with increased benefit, in terms of improved tolerability as above defined and improved efficacy.

This represents a totally surprising and unexpected re-suit compared with the known steroidal compounds. Indeed considering the various above therapeutic uses of a specific precursor product, the present invention products give a combination of results, considered as improvement of the therapeutic performance, i.e. improved pharmacotherapeutic efficacy and improved tolerability, compared with the prior art products. In fact, contrary to any expectations, the present invention products are characterized in that they show an improved therapeutic profile: high activity in the above applications combined with lower side effects as above defined.

As known, steroids comprise:
corticosteroids, classified in glucocorticoids active on the glucogenesis and on the metabolism of proteins, lipids, carbohydrates and calcium in general, mineralcorticoids active on the water and saline balance;
sexual steroids, including estrogens and androgens.

It is well known that glucocorticoids represent a first choice pharmacological approach in the therapy of various pathologies. Said class of drugs, among which, for example, hydrocortisone, cortisone, prednisone, prednisolone, fludrocortisone, desoxycorticosterone, methylprednisolone, triamcinolone, paramethasone, betamethasone, dexamethasone, triamcinolone acetonide, fluocinolone acetonide, beclomethasone, acetoxypregnelone, etc. can be mentioned, exerts marked pharmaco-toxicological effects on various organs. For said reason the prolonged clinic use and the interruption of the pharmacological treatment cause side effects, some of them very serious. See for example Goodman & Gilman, “The Pharmacological Basis of Therapeutics” 9th ed., pages 1459-1465, 1996.

Among the side effects one can mention:
those affecting the bony tissue, such as for example osteoporosis, osteonecrosis and myopathies, which in patients affected by asthma or by COPD (Chronic Obstructive Pulmunary Disease) can determine a marked reduction of the respiratory capabilities;
those affecting the cardiovascular system which generate hypertensive responses and/or cardiac frequency diseases;
increased easyness to infections;
those affecting the gastrointestinal apparatus;
increase of glucose levels in the blood, which in diabetic patients can lead to a disease worsening, or in predisposed patients it can cause the arising of hyperglycaemic attacks.

See for example Martindale “The Extrapharmacopoeia”, 30th ed., pages 712-723, 1993.

According to the prior art it does not seem possible to separate the steroid therapeutic actions from their side effects, see Goodman et al, mentioned above, page 1474.

In the prior art nitrooxy derivatives of steroids, which are usable also as cardiovascular agents for the coronary insufficiency or angina pectoris therapy, are described.

For example, German patent DE 2,222,491 describes the preparation of pregnane derivatives having in position 21 the —CH₂—O—NO₂ group. In said patent it is stated that said derivatives have a cardiotropic activity. This activity represents a drawback for said compounds, since they modify the cardiac frequency. Furthermore in said patent no mention is made to the receptor affinity on the specific receptors of the endogenous steroids.

U.S. Pat. No. 3,494,941 describes steroid derivatives from 3-hydroxy-extrane or from extr-4 en-3 one, used as vasodilators in the treatment of cardiac affections such as coronary insufficiency and angina pectoris. In the structure of said compounds a ONO₂ group is at the free end of the alkylene chain which is linked by an ether bond to the steroid in position 17. According to said patent it is possible to have nitrate groups also in the positions 3 and 16 of the steroidal structure. The same drawbacks mentioned above as regards the effects on the cardiac frequency can be repeated for the compounds of this patent. Besides, in the patent no mention is made to the receptor affinity on the specific receptors of endogenous steroids.

U.S. Pat. No. 3,183,252 describes derivatives of 16-nitrate-alkylpregnanes wherein the alkyl group is linked to the pregnane structure by a carbon-carbon bond. The compounds according to said patent can be used as vasodilators. The same drawbacks reported for the above prior art can be repeated.

WO 98/15568 in the name of the Applicant describes nitrate esters of steroidal compounds, wherein between the steroidal structure and the nitrooxy group a bivalent linking group is inserted. Said compounds show a good efficacy and/or good tolerability with respect to the corresponding precursors. However in the examples and in the description no data are reported on the receptor activity. No indication is therefore reported suggesting steroidal compounds having an improved receptor activity and an improved pharmacological activity combined with lower side effects.

Patent application WO 00/61604 in the name of the Applicant describes nitrooxy derivatives of steroidal compounds with various linking groups having at one end a nitrooxy group, and covalently linked with the other end to a steroidal compound. In said application the uses concern the compounds usable in the treatment of patients in oxidative stress. Said compounds contain in the molecule also a bivalent linking group which must be capable to prevent the free radicals production and is selected on the basis of the tests reported therein. No indication is therefore given suggesting steroidal compounds having an improved receptorial activity and an improved pharmacological activity combined with lower side effects.

The need was felt to have available steroidal compounds having an improved receptor affinity on the specific receptors of the endogenous steroids and an improved pharmacological activity combined with lower side effects. The Applicant has surprisingly and unexpectedly found a specific class of steroidal compounds which in the above pathologies unexpectedly and surprisingly show not only an improved efficacy but also an improved tolerability and lower side effects compared with the steroids of the prior art.

An object of the present invention are nitrooxy derivatives of steroidal compounds of general formula
B—X₁—NO₂ (I)
or esters or salts thereof, wherein:
B is a steroidal radical having the following structure: 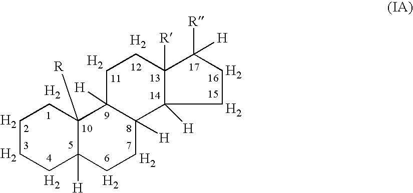
wherein at the place of the hydrogen of the CH group, or of the two hydrogens of the CH₂ group indicated in the general formula (IA), there can be the following substituents:
in position 1-2: a double bond;
in position 2-3 the following substituent: 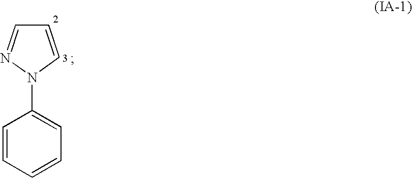
in position 2: Cl, Br;
in position 3: oxo, —O—CH₂—CH₂—Cl, OH, OCH₃;
in position 4-5: a double bond;
in position 5-6: a double bond;
in position 6: Cl, F, Br, CH₃, —CHO;
the ring defined by the carbon atoms numbered 1, 2, 3, 4, 5, and 10 is an aromatic ring when B is the residue of an estrogen;
in position 7: Cl, OH;
in position 9: Cl, F, Br;
in position 11: OH, oxo, Cl;
in position 16: CH₃, OH, ═CH₂;
in position 17: OH, CH₃, OCO(O)ᵤₐ(CH₂)ᵥₐCH₃ wherein ua is an integer equal to 0 or 1, va is an integer from 0 to 4, ethynyl (C═CH); or 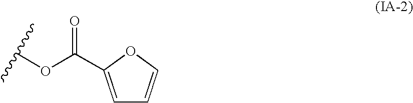
in position 16-17 the following groups: 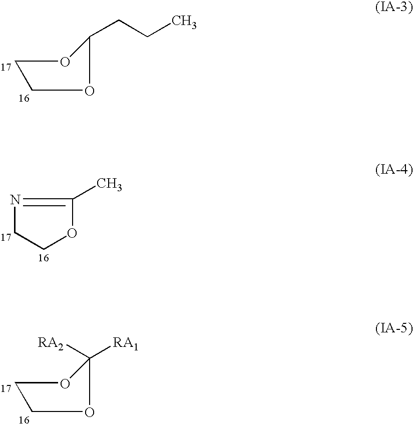
   wherein RA₁ is H, CH₃; RA₂ is a C₁-C₁₀ linear or branched alkyl chain, preferably C₁-C₃, still more preferably CH₃, or a saturated cycloaliphatic ring having 5-6 carbon atoms or an aromatic ring optionally substituted in para position with N(R_{1c})₂ wherein R_{1c} is a C₁-C₁₀, preferably C₁-C₄, linear or branched alkyl;
   R and R′, equal to or different from each other, can be hydrogen or C₁-C₄ linear or branched alkyls, preferably R═R′═CH₃ or R═R′═H;
      preferably B is a corticosteroid residue;
   R″ in position 17 is a bivalent radical having one of the following meanings:
   IB) —(CO-L)ₜ-(X_{O})ₜ₁-, wherein t=1 and t1 is 0 or 1, preferably 0;
   IC) -L-(X₀)ₜ₁-, wherein t1 is 0 or 1, preferably 1;
      the bivalent linking group L has the following meaning:
      (CR₄R₅)ₙₐ(O)_{nb}(CR_{4′}R_{5′})_{n′a}(CO)_{n′b}(O)_{n″b}(CO)_{n′″b}(CR_{4″}R_{5″})_{n″a}
      wherein na, n′a, and n″a, equal to or different from each other, are integers from 0 to 6, preferably 1-3; nb, n′b, n″b and n′″b, equal to or different from each other, are integers equal to to 0 or 1; R₄, R₅, R_{4′}, R_{5′}, R_{4″}, R_{5″}, equal to or different from each other, are selected from H, C₁-C₅, preferably C₁-C₃ linear or branched alkyl;
   Xₒ≡O—, —NH—, —NR_{1c}— wherein R_{1c} is as above defined; the bond between the steroid B and the linking group X₁ is ester or amidic type;
   X₁ is a bivalent linking group selected from the following:
   Y_{AR1}: 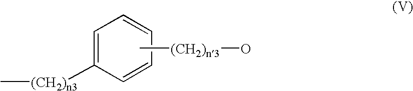
   wherein n3 is an integer from 0 to 5 and n3′ is an integer from 1 to 3;
   Y_{AR2}: 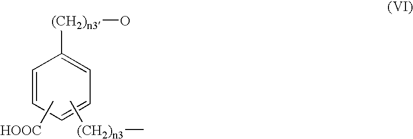
   wherein n3 and n3′ have the above meaning.
   Yₚ: 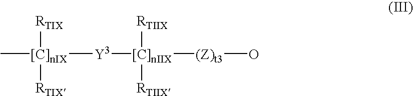
   wherein:
   nIX is an integer from 0 to 10, preferably 1-3;
   nIIX is an integer from 1 to 10, preferably 1-5;
   R_{TIX}, R_{TIX′}, R_{TIIX}, R_{TIIX′}; equal to or different from each other are H or C₁-C₄ linear or branched alkyl; preferably R_{TIX}, R_{TIX′}, R_{TIIX}, R_{TIIX′} are H;
   Y³ is a saturated, unsaturated or aromatic heterocyclic ring, having 5 or 6 atoms, containing from one to three heteroatoms, preferably from one to two, said heteroatoms being equal or different and selected from nitrogen, oxygen, sulphur; preferably nitrogen;
   t3 is zero or 1;
   Z has the following meaning: 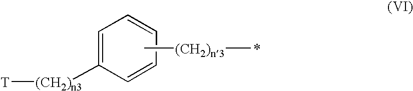
      wherein:
      * shows the position of the ONO₂ group;
      T has the following meanings:
      —COX₃—, —X₃CO—, wherein X₃═S or Xₒ as above defined;
   —X₃— as above defined;
   n3 and n′3 are as above defined.

The linking group X₁ links to the radical B with the indicated valence which does not bring the oxygen.

Preferably Y³ is selected from the following bivalent radicals:

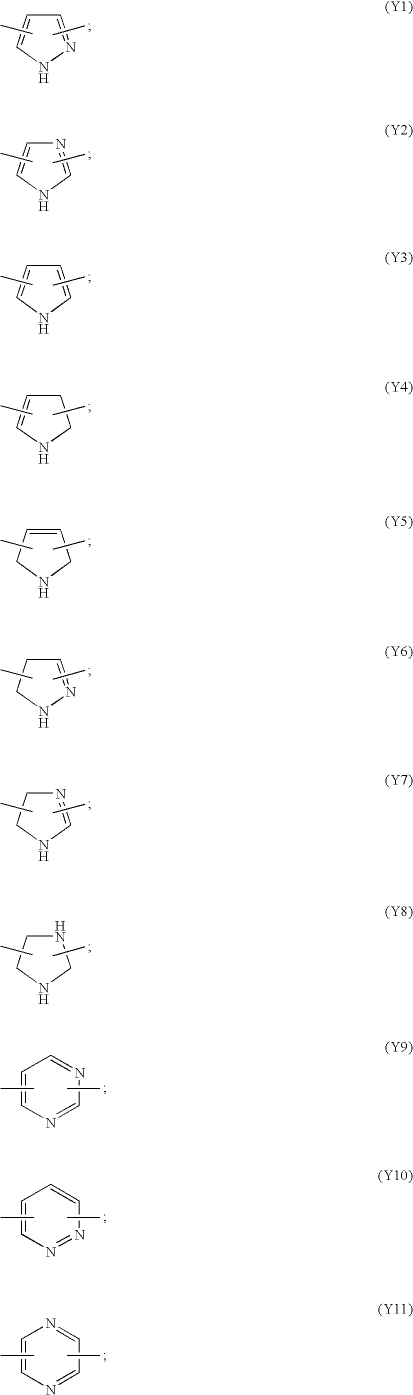

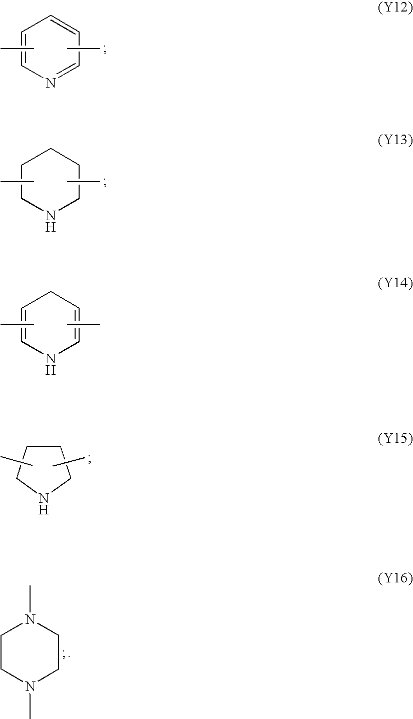

The following are the preferred of Y³: (Y12), having the two free valences in the ortho positions with respect to the nitrogen atom; (Y16) with the two valences linked to the two heteroatoms, (Y1) (pyrazol) 3,5-disubstituted; (Y16) is particularly preferred.

The invention preferred compounds are those wherein the precursor of B has the meanings mentioned below.

For example as precursors of the steroids of the present invention can be mentioned those described in the Merck Index, 12th Ed. 1996, herein integrally incorporated by reference, in which also the respective synthesis methods are mentioned, and in the patents indicated hereinafter. The precursors (according to the Merck nomenclature) are the following: corticosteroids selected from Budesonide, Hydrocortisone, Alclomethasone, Algestone, Beclomethasone, Betamethasone, Chloroprednisone, Ciclesonide (U.S. Pat. No. 5,482,934), Clobetasol, Clobetasone, Clocortolone, Cloprednol, Cortisone, Corticosterone, Deflazacort, Desonide, Desoximethasone, Dexamethasone, Dexamethasone 17-furoate, Diflorasone Diflucortolone, Difluprednate, Fluazacort, Flucloronide, Flumethasone, Flunisolide, Fluocinolone Acetonide, Fluocinonide, Fluocortin Butyl, Fluocortolone, Fluorometholone, Fluperolone Acetate, Fluprednidene Acetate, Fluprednisolone, Flurandrenolide, Formocortal, Halcinonide, Halobetasol Propionate, Halometasone, Halopredone Acetate, Hydrocortamate, Itrocinonide (EP 197,018), Loteprednol Etabonate, Meclonisone (U.S. Pat. No. 4,472,393), Medrysone, Meprednisone, Methylprednisolone, Mometasone Furoate, Paramethasone, Prednicarbate, Prednisolone, Prednisolone 25-Diethylaminoacetate, Prednisolone Sodium Phosphate, Prednisone, Prednival, Prednylidene, Rofleponide Rimexolone, Triamcinolone, 21-Acetoxy-pregnenolone, Cortivazol, Amcinonide, Fluticasone Propionate, Mazipredone, Taucorten, Tixocortol, Triamcinolone Hexacetonide; bile acids selected from Ursodesoxycholic acid, Chenodesoxycholic acid; estrogens selected from Mytatrienediol, Moxestrol, Ethynylestradiol, Estradiol, Mestranol, methyl (20R)-6-alpha, 9alpha-difluoro-11beta-hydroxy-16alpha, 17alphapropyl methylene dioxyandrosta-1,4-dien-3-one-17beta-carboxylate (EP 143,764).

Preferably when B is the residue of a corticosteroid, R″=IB with t=1 and t1=0; preferably in the formula of the linking group L na=nb=n′b=1; n′a=n″=b=n′″b=n″a=0; R₄, R₅=H; or n′b=n″b=1 and all the other na, nb, n′a, n′″b, n″a are equal to zero.

Preferably when B is the residue of a bile acid, R″=IC with t1=1; preferably in the formula of the linking group L na=n′b=1, n′a=2, n″b=n′″b=n″a=nb=0, R₄=CH₃, R₅=R_{4″}=R_{5″}=H.

Preferably when B is the residue of an estrogen then
—R″=IC wherein t1=0 and in the formula of the linking group L nb=n′b=1; na=n′a=n″b=n′″b=n″a=0, the other substituent in position 17 is preferably H or ethynyl, and in position 3 one of the substituents can optionally be the —R″—X₁—NO₂ group (R″ as above defined); or
in position 3 the —R″—X₁—NO₂ group is present, R″ being as above when B is the residue of an estrogen, in this case the substituents of the carbon atoms in position 17 in formula (IA) of B are the following:
R″═—O—, wherein the free valence of the oxygen is saturated with H;
H in the formula (IA) is substituted with a group different from OH.

The precursors of the bivalent radicals X₁ as above defined, wherein the oxygen free valence is saturated with H and the free valence of the end carbon atom is saturated either with a carboxylic or hydroxyl or amminic group, are commercial products or they can be synthesized according to known methods of the prior art.

The preferred compounds according to the present invention are the following:
Hydrocortisone 21-(4′-nitrooxymethyl) benzoate 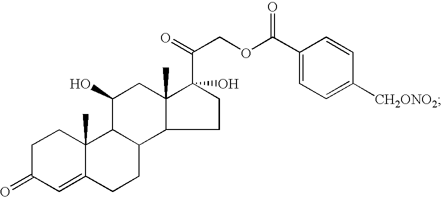
Hydrocortisone-21-[2-[6-(nitrooxymethyl)-2-pyridinyl]acetate] 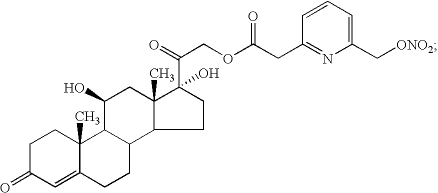
Hydrocortisone-21[2-[4-[2-[4-(nitrooxymethyl)benzoyloxy]ethyl]1-piperazinyl]acetate] 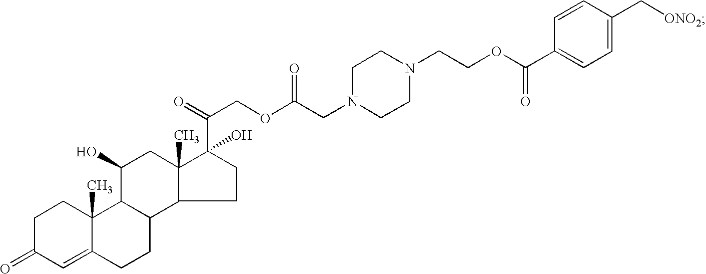
Hydrocortisone-21-[2-[4-[3-(nitrooxy)propyl]-1-piperazinyl]acetate] 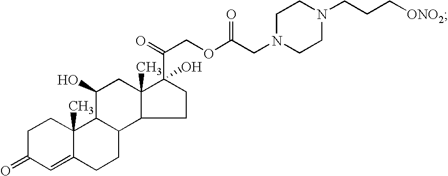
Dexamethasone 21-(4′-nitrooxymethyl)benzoate 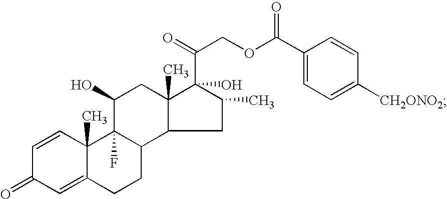
Dexamethasone-21-[2-[4-[3-(nitrooxy)propyl]-1-piperazinyl]acetate] 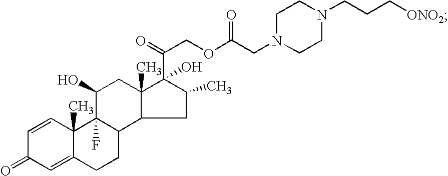
Dexamethasone-21[2-[4-[2-[4-(nitrooxymethyl)benzoyloxy]ethyl]-1-piperazinyl]acetate] 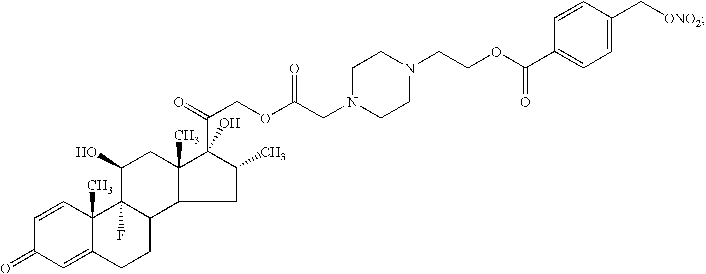
Dexamethasone-21-[2-[6-(nitrooxymethyl)-2-pyridinyl]acetate] 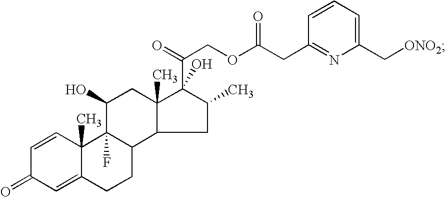
Prednisolone 21-(4′-nitrooxymethyl)benzoate 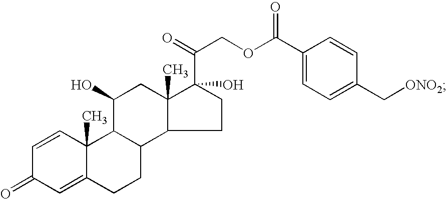
Budesonide 21-(4′-nitrooxymethyl)benzoate: 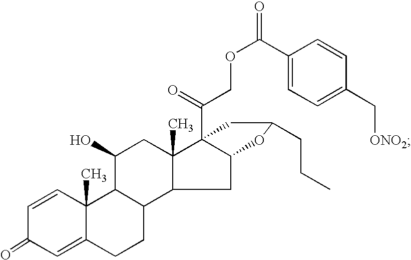
Budesonide-21-[2-[6-(nitrooxymethyl)-2-pyridinyl]acetate] 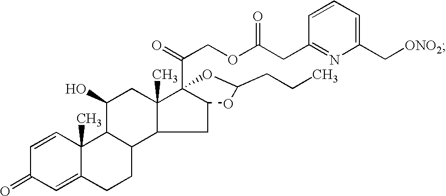
Budesonide-21-[2-[4-[3-(nitrooxy)propyl]-1-piperazinyl]acetate] 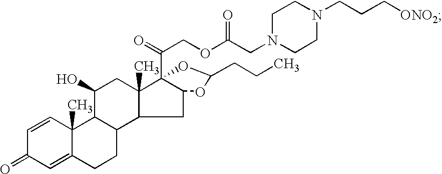
Budesonide-21[2-[4-[2-[4-(nitrooxymethyl)benzoyloxy]ethyl]-1-piperazinyl]acetate] 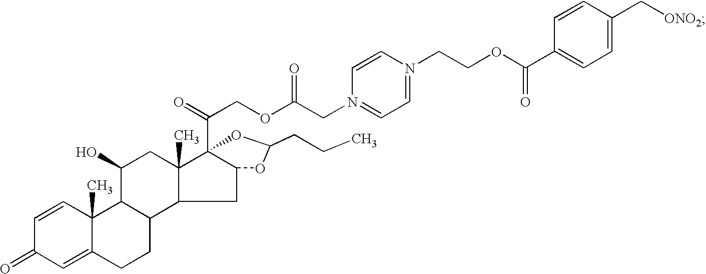
Flumethasone 21-(4′-nitrooxymethyl)benzoate: 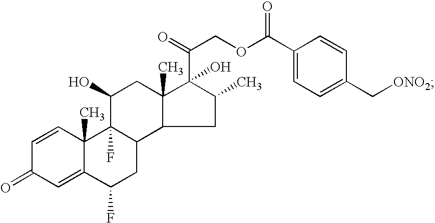
Flumethasone-21-[2-[6-(nitrooxymethyl)-2-pyridinyl]acetate] 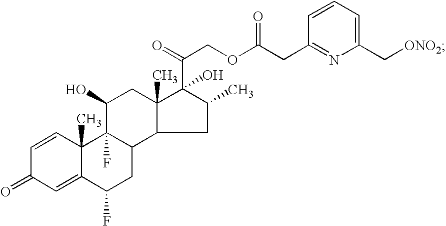
Flumethasone-21[2-[4-[2-[4-(nitrooxymethyl)benzoyloxy]ethyl]-1-piperazinyl]acetate] 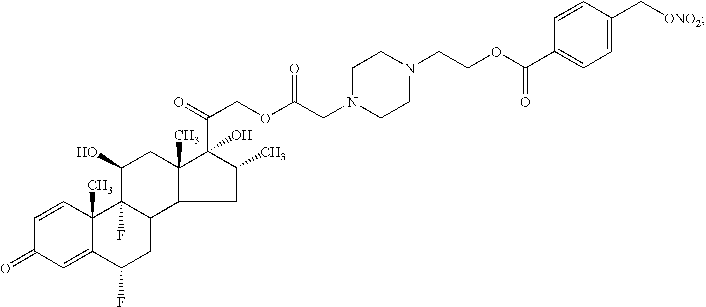
Flumethasone-21-[2-[4-[3-(nitrooxy)propyl]-1-piperazinyl]acetate] 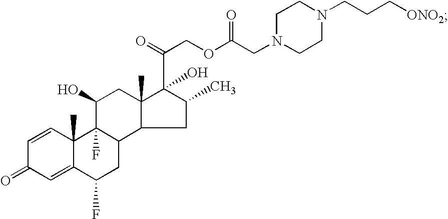
Prednisolone-21-[2-[6-(nitrooxymethyl)-2-pyridinyl]acetate] 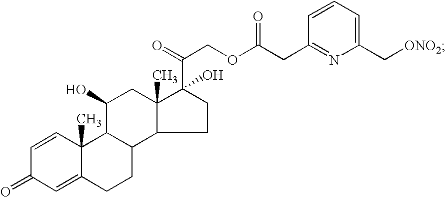
Prednisolone-21-[2-[4-(3-nitrooxy) propyl) piperazin-1-yl]acetate]and the corresponding bishydrochloride salt: 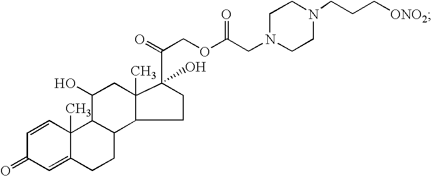
Prednisolone-21-[2-[4-[2-[(4′-nitrooxymethyl)benzoyloxy]-ethyl]piperazin-1-yl]acetate]and the corresponding bis-hydrochloride salt: 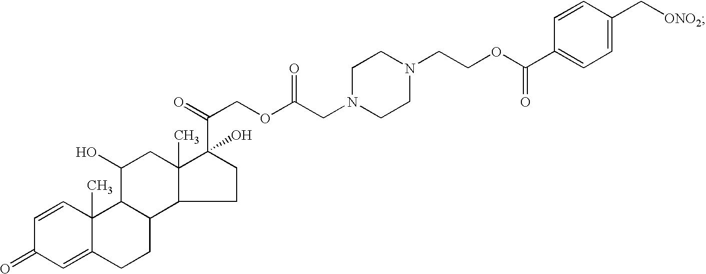
Flunisolide-21-[2-[4-[3-(nitrooxy)propyl]-1-piperazinyl) acetate] 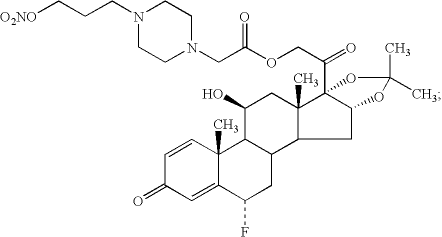
Flunisolide-21-[(4′-nitrooxymethyl)]benzoate 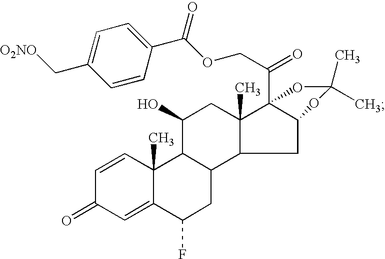
Flunisolide-21-[2-[4-[2-[4-(nitrooxymethyl)benzoyloxy]ethyl]-1-piperazinyl]acetate] 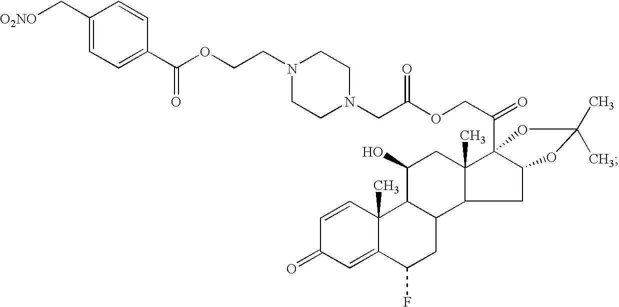
Flunisolide-21-[2-[6-nitrooxymethyl)-2-pyridinyl]acetate] 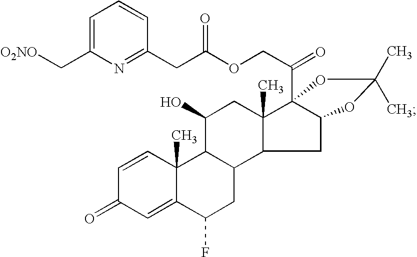
Flunisolide-21-[2-(6-(nitrooxymethyl)-2-pyridinyl]acetate]

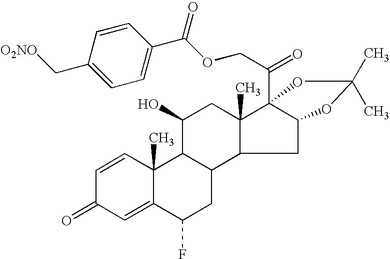

Generally the connection between B and X₁ is, as said, of ester or amidic type (NH or NR_{1C}, as defined in X₀). For the formation of said functional groups the synthesis methods described in the prior art are usable.

The compounds according to the present invention, when at least a functional group salifiable with acids, for example an amminic group, is present, can be transformed into the corresponding salts. For example one way to form the salts is the following: when one basic nitrogen atom is present in the molecule, it is reacted in organic solvent such for example acetonitrile, tetrahydrofuran, with an equimolecular amount of the corresponding organic or inorganic acid. Examples of organic acids are: oxalic, tartaric, maleic, succinic, citric, trifluoroacetic acid. Examples of inorganic acids are: nitric, hydrochloric, sulphuric, phosphoric acid.

When the precursor compounds usable in the present invention have one or more chiral cores, they can be in a racemic form or as diastereosisomer mixtures, as single enantiomers or single diastereoisomers; if they show a geometric asymmetry the compounds can be used in the cis or trans form.

When the functional group of the steroid structure which reacts with X₁ (for ex. —COOH, —OH) is bound with a covalent bond type, for example, ester, amide, ether, said function can be restored by the well known methods of the prior art.

Generally when in the reacting compounds more functional groups are present, said groups can be protected before the reactions according to the known methods of the prior art; for example as described in “Protective groups in organic synthesis”, Th. W. Greene, Harward University Press, 1980.

The acylhalides used in the invention compound synthesis can be prepared according to known methods of the prior art, for example by reacting the corresponding carboxylic acids with thionyl chloride or oxalyl chloride, with P^{III} or P^{V} halides in solvents inert under the reaction conditions.

1. If the steroid reactive function is the hydroxyl group (B—OH) and the bond between the steroid and the linking group X₁ is of the ester type, the most used synthesis methods are the following:

1a. Reaction between the steroid molecule with a compound of formula Hal-C(O)—X_{1A}-Hal wherein Hal=Cl, Br, I, and X_{1A} is a radical obtained from Y_{AR1} (formulas V and VI) or Y_{P} (formula III) with t3=0, omitting the oxygen atom —O—, in the presence of an organic base as triethylamine or pyridine, etc., using a solvent inert under the reaction conditions such as DMF, toluene, tetrahydrofuran, etc. and a temperature in the range 0° C.-25° C. according to the following scheme (IA):
B—OH+Hal-C(O)—X_{1A}-Hal→B—OC(O)—X_{1A}-Hal (IA.1)

The corresponding nitrooxy derivative is obtained by reacting the compound (IA.1) obtained from the previous reaction with AgNO₃ in an organic solvent as acetonitrile, tetrahydrofuran at a temperature in the range 25° C.-80° C., according to the following scheme (IA.2):
B—O(CO)—X_{1A}-Hal+AgNO₃→B—O(CO)—X_{1A}—ONO₂ (IA.2)
1b. Alternatively to the synthesis described in 1a, the compound HO—C(O)—X_{1A}-Hal wherein Hal and X_{1A} have the above meanings, can be treated with a carboxyl activating agent, selected from N,N-dicarbonyldiimidazol (CDI), N-hydroxy benzotriazol or dicyclohexylcarbodiimide (DCC), in an organic solvent such for example DMF, tetrahydrofuran, chloroform, etc., at a temperature in the range −5 and 50° C. The obtained compound is reacted in situ with the steroid (B—OH) to give the compound of formula (IA.1).

The corresponding nitrooxy derivative is obtained as above described.

2. If the steroid reactive function is the hydroxyl group (B—OH), when X₁=Y_{P} and in particular Y_{P}=Y16 and besides the bond between the steroid, the linking group X₁, is of ester type, t3=0 in formula (III), the usable synthesis method is for example the following:

2a. The steroid B—OH is reacted with a compound of formula Hal-C(O)-M-Hal wherein Hal=Cl, Br, I and M is a C₁-C₁₀ linear or substituted alkylene, in the presence of an organic base as triethylamine or pyridine, etc., using a solvent inert under the reaction conditions as DMF, toluene, tetrahydrofuran, etc., at a temperature in the range of 0° C.-25° C. according to the following scheme (IIA):
B—OH+Hal-C(O)-M-Hal→B—O(CO)-M-Hal (IIA.1)

The formula (IIA.1) compound is then reacted with the bishydrochloride of a N-(ω-halogenalkyl)piperazine or with N-(ω-hydroxyalkyl)piperazine, wherein the alkylene equal to or different from M is M′, in the presence of an organic base as triethylamine, etc., using a solvent inert under the reaction conditions as DMF, toluene, tetrahydrofuran, etc., and at a temperature in the range −5° C. and 0° C., according to the scheme (IIB) to give the compound (IIB.1): 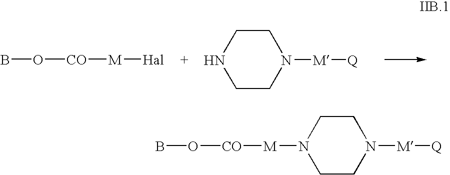
wherein M and M′ are as above; Q=OH, Cl, Br, I.

When Q=Cl, Br, I the compound (IIB.1) is reacted with AgNO₃ as indicated above to obtain the corresponding nitrooxy-derivative.

When in formula (III) t3=1, the function which is at the free end of the bivalent radical M′ is reacted according to the synthesis scheme for example reported in Ia, wherein X_{1A} is the radical of formula (VI) but omitting the T functional group. A compound having a group Hal is obtained which is reacted with AgNO₃ as above described.

3. If the steroid reactive function is the carboxyl group (R—COOH) and the bond between the steroid and the linking group X₁ is of ester type, the most used synthesis method is the following:

3a. The steroid (R—COOH) is treated with an agent activating the carboxyl selected from N,N-dicarbonyldiimidazol (CDI), N-hydroxybenzotriazol or dicyclohexylcarbodiimide (DCC) in an organic solvent such for example DMF, tetrahydrofuran, chloroform, etc., at a temperature in the range −5° C.-50° C. The obtained compound is reacted in situ with the precursor of X₁ of formula HO—X_{1A}-Hal wherein X_{1A} is a radical obtained from Y_{AR1} or Y_{P} omitting the oxygen atom —O—, and Hal is as above defined. The obtained compound, having general formula B—C(O)—O—X_{1A}-Hal, is reacted with AgNO₃ as above described to give the corresponding nitrooxy derivative.

4. If the steroid reactive function is the carboxyl group (R—COOH) and the bond between the steroid and the linking group X₁ is of amidic type, the most used synthesis method is the following:

4a. The steroid (R—COOH) is treated with an agent activating the carboxyl selected from dicyclohexylcarbodiimide (DCC) in an organic solvent as for example DMF, tetrahydrofuran, chloroform, etc., at a temperature in the range −5° and 50° C. and the obtained compound is reacted in situ with the precursor of X₁ of formula H₂N—X_{1A}-Hal wherein X_{1A} and Hal are as defined above. The obtained compound having general formula B—C(O)—NH—X^{1A}-Hal is reacted with AgNO₃ as described above to give the corresponding nitrooxy derivative.

The Applicant has unexpectedly found that the invention compounds wherein the linking group X₁ is selected from the above mentioned bivalent radicals, allow to obtain, see the examples of the receptor binding assays, results unexpectedly and surprisingly improved with respect to the nitrooxy derivatives wherein the linking group X₁ is an alkylene and/or with respect to the corresponding precursor steroids.

Said results are quite unexpected since in the prior art there is no mention that with the linking groups described in the present invention it was possible to improve the receptor binding.

It has been found that the present invention compounds do not affect the cardiocirculatory parameters and therefore the present invention compounds do not give undesired effects on the systemic pressure and on the cardiac frequency. Besides the invention compounds show an improved pharmacological activity combined with lower side effects, in particular:
affecting the bony tissue, such for example osteroporosis;
affecting the gastrointestinal apparatus.

The invention compounds have not only an improved antiinflammatory activity at a peripheral level, but also an improved anti-neurodegenerative activity, the compounds being active on the neurodegenerative diseases on an inflammatory and traumatic basis of the nervous system, such for example spinal trauma and lesions and cerebral trauma, inflammation of the nervous tracts such as the sclerosis multipla. The invention compounds show furthermore an improved antiarthritic activity, improved immunodepressive activity, improved angiostatic/angiogenetic and antiasthmatic activity.

The invention compounds are usable in substitutive hormonal therapies, for example in the post-menopause therapy. The compounds according to the present invention are therapeutically useful in the treatment of morbid conditions wherein steroidal precursor products are used, but with increased benefit, in terms of improved tolerability as defined above and improved efficacy. As a matter of fact, contrary to any expectations the present invention products are characterized in that they show an improved therapeutic profile: high activity in the above applications combined with lower side effects as defined above. It has been unexpectedly found that the invention compounds show lower side effects, in particular as regards:
those affecting the bony tissue, such for example osteoporosis, osteonecrosis and myopathies, which in patients affected by asthma or by COPD (Chronic Obstructive Pulmunary Disease) can determine a remarkable reduction of the respiratory capabilities;
those affecting the cardiovascular system which generate hypertensive responses and/or cardiac frequency diseases;
lower predisposition to infections;
those affecting the gastrointestinal apparatus.

The compounds object of the present invention are formulated in the corresponding pharmaceutical compositions, also with belated release, for parenteral, oral and topic use, such as for example sublingual, inhalatory, suppository, transdermal, enema, according to the well known techniques in the art, together with the usual excipients; see for example the publication “Remington's Pharmaceutical Sciences” 15th Ed.

The amount on a molar basis of the active principle in said compositions is generally the same, or lower than that of the corresponding precursor drug.

The daily administrable doses are those of the precursor drugs, or optionally lower. The precursor daily doses can be found in the publications of the field, such for example in the “Physician's Desk reference”.

The present invention compounds are used for the treatment of pathologies wherein the precursor steroids are used. In particular, the use is mentioned as drugs in rheumatic diseases, renal and bronchial pathologies, ocular and dermatological diseases, autoimmune diseases, tumoral processes, also in combination with chemotherapeutic and/or radiotherapeutic treatments, in neurodegenerative diseases, for example in spinal lesions from trauma and in the post-transplant therapy. Furthermore inflammatory pathologies affecting the gatrointestinal system (Crohn disease, ulcerous colitis and IBD (inflammatory bowel diseases) can be mentioned.

Further, the Applicant has surprisingly and unexpectedly found that the invention steroids of the glucocorticoid class can be used, differently from precursors, in respiratory pathologies characterized by broncho-obstructive events. Said fact is quite unexpected since the precursors are substantially ineffective under said morbid conditions; indeed they must be associated with broncho-dilators as beta-agonists such for example salbutamol.

For said use as bronchodilators the Applicant has found that not only the nitrooxyderivatives of the steroids according to the present invention are effective, but also the derivatives in which the linking group X₁ in formula (I) is an aliphatic linking group of the glucocorticoid class of formula (I), selected from the following:
I) An alkylenoxy group R′O wherein R′ is C₁-C₂₀— linear or branched when possible, preferably having from 2 to 6 carbon atoms, or a cycloalkylene having from 5 to 7 carbon atoms, in the cycloalkylene ring one or more carbon atoms can be substituted by heteroatoms, the ring can have side chains of R′ type, R′ being as above defined; II) or one of the following groups: 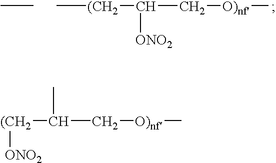
   wherein nf′ is an integer from 1 to 6 preferably from 1 to 4; 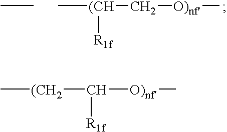
   wherein R_{1f}=H, CH₃ and nf′ is an integer from 1 to 6;
preferably from 1 to 4.

For said use both the invention compounds with the linking groups as defined above and those wherein the linking groups are selected from those indicated in I) or in II), can be used.

The present invention compounds, differently from the precursors, have no side effects on the bony system, in particular they do not cause bony reabsorption, besides they show a high gastric tolerability.

The following Examples are given for illustrative purposes but they are not limitative of the present invention.

### EXAMPLE 1

### Comparative

### Synthesis of Prednisolone 21-(4-nitrooxy)butyrate

### A. Prednisolone 21-(4-chlorobutyrate)]

To a solution of prednisolone (2.5 g, 7 mmoles) in tetrahydrofuran (150 ml), triethylamine (3.9 ml) and 4-chlorobutyryl chloride (5.2 g), in the order, have been added. The solution has been kept under stirring at room temperature for 4 hours. The solvent has been removed by evaporation under vacuum. The raw residue has been extracted with a mixture of ethyl acetate and water. The organic phases have been joined, dried with sodium sulphate, then concentrated at reduced pressure. The obtained residue has been crystallized by hexane/ethyl acetate. The product has been isolated as a yellow solid (2.9 g).

### B. Prednisolone 21-[(4-nitrooxymethyl)butyrate]

A solution formed by prednisolone 21-(4-chlorobutyrate) (2.8 g, 5.5 mmoles) and silver nitrate (1.87 g, 11 mmoles) in acetonitrile (130 ml) and tetrahydrofuran (30 ml) has been prepared, then refluxed, sheltered from the light for 18 hours. The precipitate, formed by silver salts has been filtered and the solvent evaporated under vacuum. The obtained residue has been purified by chromatography on silica gel, eluent hexane/ethyl acetate (6/5 v/v). The product has been crystallized by tetrahydrofuran/n-hexane to give 1.1 g of a white solid.

M.p.: 80°-85° C.

¹H-NMR (200 MHz, DMSO) ppm: 7.38 (1H,d); 6.22 (1H,dd); 5.95 (1H,s); 5.45 (1H,s); 5.16(1H,d); 4.84 (1H,d); 4.76 (1H,d); 4.65 (2H,t); 4.35 (1H,s); 3.35 (2H,m); 2.58 (5H, m); 2.35 (1H,m); 2.15-0.90 (12H,m); 1.42 (3H,s); 0.82 (3H,s).

### EXAMPLE 2

### Sinthesis of hydrocortisone 21-(4′-nitrooxymethyl)benzoate

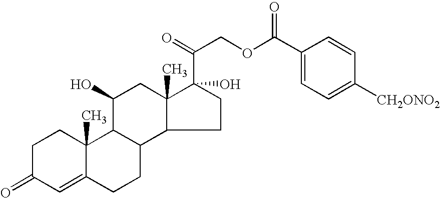

### A. Hydrocortisone 21-[(4′-chloromethyl)benzoate]

To a solution of 0.5 g of hydrocortisone in tetra-hydrofuran (20 ml) triethylamine (0.192 ml) and 4-chloro-benzoyl chloride (0.26 g) have been added. The solution has been kept under stirring at room temperature for 24 hours. The solvent has then been evaporated under vacuum. The obtained raw residue has been extracted with a mixture of water and ethyl acetate. The organic phases have been joined, dried with sodium sulphate and concentrated at reduced pressure. The residue has been purified by chromatography on silica gel column, eluting with methylene chloride/acetone 9/1. 0.6 of a solid compound at room temperature are obtained.

### B. Hydrocortisone 21-[(4′-nitrooxymethyl)benzoate]

A solution of 2.33 g (4.57 mmoles) of hydrocortisone 21-[(4′-chloromethyl) benzoate]and silver nitrate (2.39 g) in acetonitrile (90 ml) and tetrahydrofuran (70 ml) has been heated at the temperature of 40° C. sheltered from the light. An amount of silver nitrate equal to 0.77 g has been added once a day for 5 days. The formed precipitate (silver salts) is filtered and the solvent evaporated under vacuum. The residue has been purified by chromatography on silica gel, eluent n-hexane/ethyl acetate 6/4. Finally 2 g of a white solid are isolated.

M.p.: 209° C., by DSC.

¹H-NMR (200 MHz, DMSO) ppm: 8.09(2H,d); 7.69(2H,d); 5.74(2H,s); 5.62(1H,s); 5.54(1H,s); 5.45-5.05(2H,dd); 4.42(1H,m); 4.35(1H,m); 2.60-0.9(23H,m).

### EXAMPLE 3

### Synthesis of Dexamethasone 21-(4′-nitrooxymethyl)benzoate

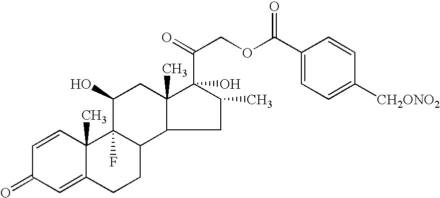

### A. Dexamethasone 21-[(4′-chloromethyl)benzoate]

To a solution of 5 g (12.74 mmoles) of dexamethasone in tetrahydrofuran (100 ml), triethylamine (1.77 ml) and 4-(chloromethyl)benzoyl chloride (2.4 g) are added. The solution is kept under stirring at room temperature for 24 hours.

After 24 hours the same above mentioned amounts of triethylamine and of acyl chloride have been added. Lastly the solvent is evaporated under vacuum. The raw residue has been extracted with a mixture of ethyl acetate and water. The joined organic phases have been dried with sodium sulphate and then concentrated at reduced pressure. The residue has been purified by chromatography on silica gel, eluent methylene chloride/acetone 9/1. A white solid (6.19 g) is obtained.

### B. Dexamethasone 21-[(4′-nitrooxymethyl)benzoate]

A solution of 6.19 g (11.35 mmoles) of dexamethasone 21-[(4′-chloromethyl)benzoate]and silver nitrate (2.89 g, 17.03 mmoles) in acetonitrile (100 ml) has been heated to 40° C. for 190 hours sheltered from the light. It is filtered, the filtrate is recovered and the solvent is evaporated under vacuum. The residue has been purified by chromatography on silica gel, eluent n-hexane/ethyl acetate 6/4. The solid has been crystallized with tetrahydrofuran/ethyl ether. The product (4.22 g) has been obtained as a white solid. M.p.: 176.8° C.

¹H-NMR (300 MHz, DMSO) ppm: 8.10(2H,d); 7.69(2H,d); 7.27 (1H,d); 6.25(1H,d); 6.07(1H,s); 5.79(1H,d); 5.74(1H,s); 5.69(1H,d); 5.38(1H,d); 5.29(1H,s); 5.14(1H,d); 4.23(1H,m); 3.96(1H,m); 2.76-2.103(1H,m); 1.90-1.32(7H,m); 1.14-0.87 (7H,m).

### EXAMPLE 4

### Synthesis of Prednisolone 21-(4′-nitrooxymethyl)benzoate

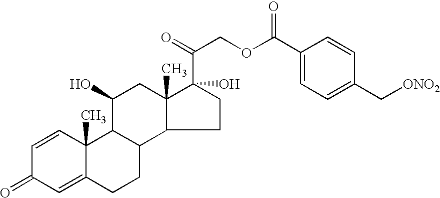

### A. Prednisolone 21-[(4′-chloromethyl)benzoate)

To a solution of 12 g (33.29 mmoles) of prednisolone in tetrahydrofuran (230 ml), triethylamine (4.64 ml) and 4-(chloro methyl)benzoyl chloride (6.29 g) are added. The solution is kept under stirring at room temperature and after one day the solvent has been evaporated under vacuum. The raw residue has been extracted with a mixture of ethyl acetate and water. The joined organic phases have been dried with sodium sulphate and then concentrated at reduced pressure. The obtained residue has been purified by chromatography on silica gel using as eluent methylene chloride/acetone 8/2. The product (16.53 g) has been obtained as a white solid.

### B. Prednisolone 21-[(4′-nitrooxymethyl)benzoate]

A solution of 16 g (31.19 mmoles) of prednisolone 21-[(4′-chloromethyl)benzoate]and silver nitrate (7.42 g, 43.66 mmoles) in acetonitrile (100 ml) and tetrahydrofuran (200 ml) has been heated under reflux sheltered from the light for 35 hours. The formed precipitate (silver salts) has been filtered and the solvent evaporated under vacuum. The obtained residue is purified by chromatography on silica gel, eluent chloroform/acetone/tetrahydrofuran 4/1/1. The product (13.3 g) has been crystallized with tetrahydrofuran (450 ml)/n-hexane (250 ml). 10.34 g of a white solid have been obtained.

M.p.: 232.5° C. by DSC.

¹H-NMR (200 MHz, DMSO) ppm: 8.15(2H,d); 7.75(2H,d); 7.45 (1H,d); 6.28(1H,dd); 6.04(1H,s); 5.80(2H,s); 5.46(1H,d); 5.16(1H,d); 4.43(1H,m); 2.63-1.06(13H,m); 1.47(3H,s); 0.95 (3H,s).

¹³C-NMR (200 MHz, DMSO) ppm: 205.298; 185.594; 171.023; 164.962; 157.118; 138.099; 129.759; 129.126; 127.023; 121.580; 88.725; 74.096; 68.362; 55.449; 51.168; 47.265; 43.916; 33.992; 33.146; 31.453; 30.955; 23.554; 20.878; 16.560.

### EXAMPLE 5

### Synthesis of Budesonide 21-(4′-nitrooxymethyl)benzoate

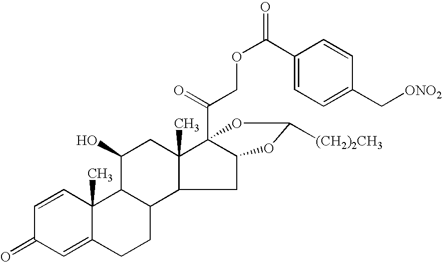

### A. Budesonide 21-[(4′-chloromethyl)benzoate]

To a solution of budesonide (5 g) in tetrahydrofuran (100 ml) triethylamine (1.62 ml) and 4-(chloro-methyl)benzoyl chloride (2.19 g) are added. The solution has been kept under stirring at room temperature and after 4 hours the same above mentioned amounts of triethylamine and acyl chloride have been added. The solution has been kept under stirring at room temperature for further 24 hours. Lastly the solvent has been removed by evaporation under vacuum and the obtained residue has been extracted with a mixture of ethyl acetate and water. The organic phases have been dried with sodium sulphate and then concentrated at reduced pressure. The obtained residue has been purified by chromatography on silica gel, eluent methylene chloride/acetone 10/1. The product (6.53 g) has been obtained as a white solid.

M.p.: 106°-110° C.

### B. Budesonide 21-[(4′-nitrooxymethyl)benzoate]

A solution of budesonide 21-[(4′-chloromethyl)benzoate](6.5 g) and silver nitrate (3.8 g) in acetonitrile (100 ml) has been heated under reflux sheltered from the light for 25 hours. The formed precipitate (silver salts) is removed by filtration and the solvent evaporated under vacuum. The residue is purified by chroamtography on silica gel, eluent n-hexane/ethyl acetate 6/4 v/v. The product (4.65 g) has been obtained as a white solid.

M.p.: 96° C. (DSC).

¹H-NMR (300 MHz, DMSO) ppm: 8.05(2H,d); 7.63(2H,d); 7.32 (1H,m); 6.17(1H,d); 5.91(1H,s); 5.67(2H,d); 5.31-5.00(2H,m); 4.75-4.72(1H,m); 4.34(1H,m); 2.51(2H,m); 2.26(2H,m); 1.98-1.94(4H,m); 1.59-1.54(4H,m); 1.39-1.35(5H,m); 0.96-0.85(7H,m).

### EXAMPLE 6

### Comparative

### Synthesis of Budesonide 21-(4-nitrooxy)butyrate

### A. Budesonide 21-(4′-chlorobutyrate)]

To a solution of Budesonide (1 g, 2.32 mmoles) in tetra-hydrofuran (20 ml), triethylamine (0.32 ml) and 4-bromobutyryl chloride (0.27 ml) have been added, in the order. After 5 hours triethylamine and the acyl chloride are added in the same above amounts. The solution has been kept under stirring at room temperature for 16 hours. The same phases of the process described in Example 1A (comparative) are repeated. The product has been isolated as a white solid (1.18 g).

### B. Budesonide 21-[(4-nitrooxymethyl)butyrate]

A solution formed by Budesonide 21-(4′-bromobutyrate) (1.18 g, 2.03 mmoles) and silver nitrate (0.52 g, 3.04 mmoles) in acetonitrile (50 ml) and tetrahydrofuran (30 ml) has been prepared, then refluxed sheltered from the light for 48 hours. The precipitated formed by silver salts has been filtered and the solvent evaporated under vacuum. The obtained residue has been purified by chromatography on silica gel, eluent methylene chloride/ethyl acetate (6/5 v/v). 850 mg of a white solid have been obtained.

M.p.: 142.5°-144.5° C.

¹H-NMR (300 MHz, DMSO) ppm: 7.32(1H,dd); 6.15(1H,d); 5.92(1H,s); 5.2-5.1(1H,m); 5.02(1H,m); 4.84(1H,m); 4.7(1H,m); 4.7(1H,m); 4.58(1H,m); 4.56(2H,t); 4.3(1H,m); 2.55(2H,t); 2.3(1H,m); 2.2-0.9(16H,m); 1.39(3H,s); 0.87 (6H,m).

### EXAMPLE 7

### Synthesis of Flumethasone 21-(4′-nitrooxymethyl)benzoate

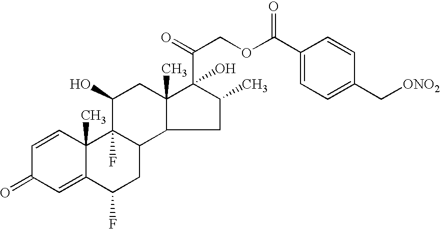

### A. Flumethasone 21-[(4′-chloromethyl)benzoate]

To a solution of Flumethasone (1 g, 2.43 moles) in tetrahydrofuran (40 ml), triethylamine (0.34 ml) and 4-(chloromethyl)benzoyl chloride (0.46 g) are added. The solution has been kept under stirring at room temperature and after 24 hours the same above amounts of triethylamine and acyl chloride have been added. The solution has been kept under stirring at room temperature for further 24 hours. Then the process described in Example 5 is repeated. The product (0.52 g) has been obtained as a white solid.

### B. Flumethasone 21-[(4′-nitrooxymethyl)benzoate]

A solution of Flumethasone 21-[(4′-chloromethyl)benzoate](0.47 g) and silver nitrate (0.21 g) in acetonitrile (100 ml) has been heated at 40° C. sheltered from the light for 30 hours. The formed precipitate (silver salts) is removed by filtration and the solvent evaporated under vacuum. The residue is purified by chromatography on silica gel, eluent n-hexane/ethyl acetate 1/1 v/v. The product (0.2 g) has been obtained as a white solid.

M.p.: 115°-120° C.

¹H-NMR (300 MHz, DMSO) ppm: 8.02(2H,d); 7.61(2H,d); 7.26(1H,m); 6.30(1H,d); 6.10(1H,s); 5.66(2H,s); 5.51(1H,m); 5.30(1H,d); 5.26(1H,s); 5.07(1H,d); 4.20(1H,m); 2.90(1H,m); 2.20(3H,m); 1.80-1.60(6H,m); 1.48(3H,s); 0.91(3H,s); 0.83(3H,d).

### EXAMPLE 8

### Synthesis of Prednisolone-21-[2-[4-(3-nitrooxypropyl) piperazin-1-yl]acetate]

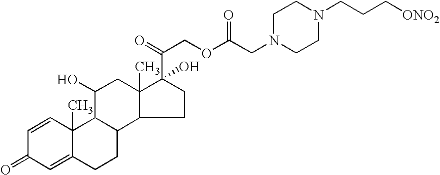

### A. Synthesis of chloroacetyl-prednisolone

To a solution of Prednisolone (1 mmole, 360 mg) in 10 ml of anhydrous THF, TEA (1.1 mmoles, 153 μl) is added. The system is cooled in a water and ice bath and chloroacetylchloride (1.1 mmoles, 87 μl) is cold added. The reaction mixture is brought to room temperature (23° C.) and maintained under stirring for 3 hours. The reaction mixture is diluted with AcOEt (10 ml) and water (10 ml). The two phases are separated: the organic phase is treated with brine (5 ml), anhydrified and dried. The obtained residue is precipitated with petroleum ether: after filtration 420 mg of product (yield 95%) are obtained as a light brown solid. The product is used as such for the successive reaction without further purifications.

### B. Synthesis of N′-t-butoxycarbonyl-N-(3-chloropropyl) piperazine

N-t-butoxycarbonylpiperazine (3 mmoles, 558 mg) (prepared according to the procedure described by Boschi D. et Al. Arch. Pharm. 1994, 327, 661-667) is dissolved in 15 ml of anhydrous CH₂Cl₂, and to said solution TEA (3.3 mmoles, 0.46 ml) is added and it is brought to 0° C. 1-bromo-3-chloropropane (3.3 moles, 0.32 ml) is cold added, it is brought under reflux (50° C.). After 3 hours the same amount of TEA and of 1-bromo-3-chloropropane is added and the reaction is maintained under reflux for 24 hours. The solvent is removed at reduced pressure, the raw product is dissolved in CH₂Cl₂ (20 ml), washed with water (10 ml). The organic phase is washed with brine (10 ml), anhydrified, the solvent removed at reduced pressure and the residue purified by chromatography on silica-gel, using AcOEt: petroleum ether 8:2 (v/v) as eluent. 470 mg of product (yield 60%) have been obtained as a very thick oil.

### C. Synthesis of N-3-chloropropylpiperazine bishydrochloride

7.5 mmoles of N′-t-butoxycarbonyl-N-(3-chloropropyl) piperazine (1.96 g) are dissolved at 0° C. in a HCl/AcOEt (50 ml) mixture. The system is left in a water and ice bath for 1 hour, then another hour at room temperature (23° C.): the formation of a white precipitate is noticed. This time elapsed the solvent is removed at reduced pressure, it is treated with ethyl ether and filtered. 1.76 g of product (m.p. 237°-239° C.) are obtained which is used without further purifications for the successive reaction.

### D. Synthesis of Prednisolone-21-[2-[4-(3-chloropropyl) piperazin-1-yl]acetate]

To a solution of chloroacetylprednisolone (1 mmole, 437 mg) in 5 ml of anhydrous DMF, the bis-hydrochloride of N-3-chloropropylpiperazine (1.2 mmoles, 282 mg) is added. The mixture is cooled to 0° C. in an ice bath and TEA (4 mmoles, 0.56 ml) is added. The mixture is left under stirring at room temperature for 18 hours, then the mixture is poured into water (5 ml) and extracted with AcOEt (2×10 ml). The joined organic extracts are washed with brine, anhydrified and dried. The so obtained yellow oily residue is purified by chromatography on silica gel eluting first with AcOEt and then with the mixture AcOEt/MeOH 9.5:0.5 (v/v).

The product obtained after the chromatographic purification is crystallized with ethyl ether, obtaining 310 mg of product (55% yield) as a yellow solid.

### E. Synthesis of Prednisolone-21-[2-[4-(3-nitrooxypropyl) piperazin-1-yl]acetate]

Prednisolone-21-[2-[4-(3-chloropropyl)piperazin-1-yl]acetate](0.55 mmoles, 310 mg) is dissolved in 8 ml of anhydrous CH₃CN and 6 ml of anhydrous THF, and to said solution AgNO₃ (1.65 mmoles, 280 mg) is added and it is brought under reflux (100° C.) under nitrogen, sheltered from the light for 5 hours. It is filtered and the solvent is evaporated at reduced pressure. The residue is purified by chromatography on silica gel using an eluent mixture of AcOEt/MeOH 9:1 (v/v). 155 mg of product have been obtained as a brown solid (48% yield).

M.p.: 116°-118° C.

¹H-NMR (300 MHz, DMSO) ppm: 7.33 (1H,d); 6.16(1H,d); 5.92(1H,s); 5.1(1H,d); 4.8(1H,d); 4.7(1H,s); 4.6(2H,t); 4.3(1H,s); 4.2(2H,t); 3.5(2H,t); 2.44(10H,s); 2.3-1.62(13H,m); 1.4(3H,s); 0.9 (3H,s).

### EXAMPLE 9

### Synthesis of Prednisolone-21-[2-[4-(3-nitrooxypropyl) piperazin-1-yl]acetate]bishydrochloride

The compound isolated at the end of Example 8 (50 mg) is dissolved in 6 ml of a mixture MeOH/DCM (dichloromethane) (1:1). To the solution cooled at 0° C. some drops of a HCl/MeOH solution are added. After 5 minutes at 0° C. the solvent is removed at reduced pressure and the residue is treated with ethyl ether. A white solid is formed which is filtered.

M.p.: >240° C.

Elemental analysis:

| | C % | H % | N % | Cl % |
|---|---|---|---|---|
| Theoretic | 54.6 | 6.83 | 6.33 | 10.7 |
| Found | 54.4 | 6.9 | 6.25 | 10.85 |

### EXAMPLE 10

### Synthesis of Prednisolone-21-[2-[4-[2-[(4′-nitrooxy methyl) benzoyloxy]ethyl]piperazin-1-yl]acetate]

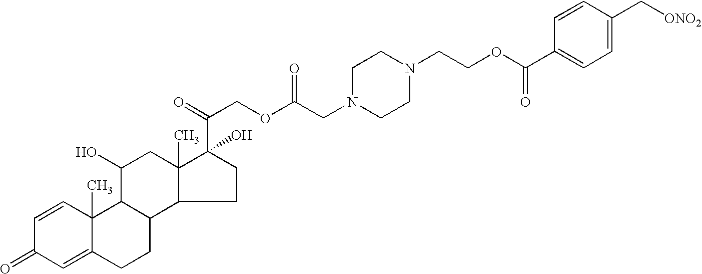

### A. Synthesis of prednisolone-21-[2-[4-(2-hydroxyethyl)-piperazin-1-yl]-acetate]

To a solution of chloroacetylprednisolone (1.5 mmoles, 660 mg) in 15 ml of anhydrous THF, N-2-hydroxyethylpiperazine (15 mmoles 1.95 g) dissolved in 15 ml of anhydrous THF is cold added (water and ice bath). After 30 minutes the mixture is brought to room temperature and after 18 hours it is filtered and the solvent is removed at reduced pressure. The residue is purified by chromatography on silica gel by using first a mixture DCM-MeOH 9:1 (v/v) then a mixture DCM-MeOH in a ratio 8:2 (v/v).

### B. Synthesis of Prednisolone-21-[2-[4-[2-[(4′-chloromethyl) benzoyloxy]ethyl]piperazin-1-yl]acetate]

The compound isolated at the end of the previous step (570 mg, 1.1 mmoles) is dissolved in 10 ml of a mixture acetonitrile/THF (4:1 v/v) and to the solution, cooled at 0° C., TEA (0.3 ml, 2.15 mmoles) and p-chloro-methylbenzoyl chloride (233 mg, 1.18 mmoles) are added. The reaction mixture is brought to room temperature, it is dried after 3 hours, the residue is treated with water (5 ml) and DCM (3×10 ml). The joined organic extracts are washed with brine (5 ml), anhydrified by Na₂SO₄ and dried. From the raw product purified by flash-chromatography (DCM/MeOH 9.5/0.5) 731 mg of product (80% yield) are recovered as a white solid.

M.p.: 215°-217° C.

### C. Synthesis of Prednisolone-21-[2-[4-[2-[(4′-nitrooxy methyl) benzoyloxy]ethyl]piperazin-1-yl]acetate]

Prednisolone-21-[2-[4-(4′-chloromethylbenzoyloxy)propyl piperazin-1-yl]acetate](0,82 mmoles, 560 mg) is dissolved in a mixture formed by anhydrous CH₃CN (16 ml) and anhydrous THF (12 ml). AgNO₃ (24.6 mmoles, 418 mg) is added. The mixture is heated under reflux shletered from the light for 3 hours. It is filtered and the solvent is removed at reduced pressure. The residue is purified by chromatography on silica gel, using a mixture AcOEt/MeOH 9/1 (v/v). 560 mg of product (96% yield) have been obtained.

M.p.: 206°-208° C.

¹H-NMR (300 MHz, DMSO) ppm: 8.0(2H,d); 7.61(2H,d); 6.16(1H,d); 5.67(2H,s); 5.41(1H,s); 5.1(1H,d); 4.82(1H,d); 4.2(1H,s); 4.4 (2H,t); 4.3(1H,s); 3.5(2H,t); 2.7-2.5(10H,m); 2.3-1.6(13H,m); 1.39(3H,s); 0.8(3H,s).

### EXAMPLE 11

### Synthesis of Prednisolone-21-[2-[4-[2-[(4′-nitrooxy methyl) benzoyloxy]ethyl]piperazin-1-yl]acetate]bishydrochloride

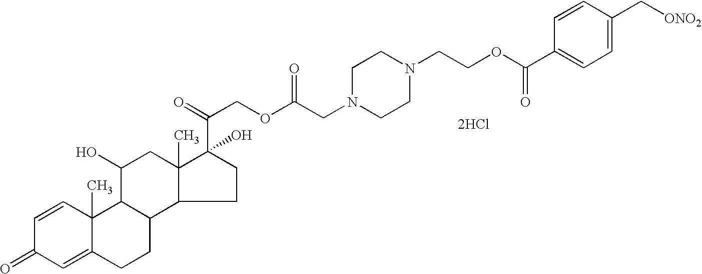

Prednisolone-21-[2-[4-(4′-nitrooxymethylbenzoyloxy) propyl piperazin-1-yl]acetate]50 mg are dissolved in 6 ml of a mixture MeOH/DCM (dichloromethane)(1:1) and to the solution cooled at 0° C. some drops of a HCl/MeOH solution are added. After 5 minutes the formed precipitate is filtered obtaining a white solid.

Elemental analysis:

| | C % | H % | N % | Cl % |
|---|---|---|---|---|
| Theoretic | 56.7 | 6.30 | 5.36 | 9.05 |
| Found | 56.6 | 6.40 | 5.25 | 9.15 |

### EXAMPLE 12

### Synthesis of Flunisolide 21-[(4′-nitrooxymethyl)benzoate)]

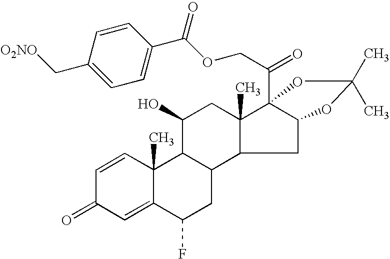

### A. Flunisolide 21-[(4′-chloromethyl)benzoate]

To a solution of 1.48 g of flunisolide in tetrahydrofuran (50 ml), triethylamine (0.71 ml) and 4-(chloro methyl)benzoyl chloride (0.96 g) are added. The solution is kept under stirring at room temperature and after one day triethylamine (0.23 ml) and 4-(chloro methyl)benzoyl chloride (0.32 g) are added. After 48 hours the solvent is evaporated under vacuum. The raw residue has been extracted with a mixture of ethyl acetate and water. The joined organic phases have been dried with sodium sulphate and then concentrated at reduced pressure. The obtained residue has been purified by chromatography on silica gel by using as eluent methylene chloride/acetone 8/2. The product (1.08 g) has been obtained as a white solid.

### B. Flunisolide 21-[(4′-nitrooxymethyl)benzoate]

A solution of 1.07 g of flunisolide 21-[(4′-chloromethyl)benzoate]and silver nitrate (0.62 g) in acetonitrile (50 ml) and tetrahydrofuran (20 ml) has been heated to 60° C. under reflux sheltered from the light for 20 hours. In the three successive days the reaction mixture is maintained under the same conditions and every day an amount of silver nitrate equal to an equivalent (0.31 g) is added, the formed precipitate (silver salts) has been filtered and the solvent evaporated under vacuum. The obtained residue is purified by chromatogrpahy on silica gel, eluent methylene chloride/ethyl acetate 9/1. The product (0.5 g) has been crystallized by methylene chloride/n-hexane. 0.4 g of a white solid have been obtained.

M.p.: 223°-225° C.

¹H-NMR (300 MHz, DMSO) ppm: 8.05(2H,d); 7.65(2H,d); 7.30 (1H,d); 6.25(1H,d); 6.03(1H,s); 5.72(1H,d); 5.69(2H,s); 5.40-5.60(2H,m); 4.91(2H,d); 4.88(1H,dd); 2.4(1H,m); 2.20(1H,m); 1.87(2H,s); 1.57-1.00(5H,m); 1.42(3H,s); 1.39(3H,s); 1.22 (3H,s); 0.88 (3H,s).

### EXAMPLE 13

### Syntheis of Prednisolone-21-[2-[4-(3-nitrooxypropyl) piperazin-1-yl]acetate]bis-trifluoromethanacetate

The compound isolated at the end of Example 8 (50 mg) is dissolved in 5 ml of acetonitrile. To the solution cooled at 0° C. some drops of a trifluoroacetic acid solution (0.4 ml) in acetonitrile (4 ml) are added. After 5 minutes at 0° C. the solvent is removed at reduced pressure and the residue is treated with ethyl ether. A white solid is formed which is filtered.

Elemental analysis:

| | C % | H % | N % | F % |
|---|---|---|---|---|
| Theoretic | 49.94 | 5.54 | 5.14 | 13.94 |
| Found | 49.89 | 5.50 | 5.18 | 13.92 |

### Pharmacological Examples

Receptor Binding Experiments

The interactioon between the steroid molecules with specific receptor proteins located in the target organ tissues, determines the receptor activation and causes a series of biochemical and physiological transformations inside the tissues, which are the steroid pharmacological effect.

The capability of a substance to bind itself to a specific receptor (affinity) and to activate the receptor itself (efficacy) is therefore a measure of its pharmacological activity.

The nitrooxyderivative efficacy according to the present invention and the corresponding nitrooxyderivatives having an aliphatic linking group, has been determined in a binding model to a glucocorticoid receptor.

In these experiments human monocytes having on their surface receptors for glucocorticoids have been used.

The corticosteroid binding itself to the receptor activates the human membrane protein CD163, isolated and characterized by Morganelli P. M. et al., J. Immunol., 1988, 140, 2296-2304.

The activation of said membrane protein depends on the pharmacological response mediated by corticosteroids. (Resnick D., et al., 1994 Trends Biochem. Sci. 19, 5-8; Hogger P. et al., J. Immunol., 1998, 161, 1883-1890; Hogger P. et al., Pharm. Res., 15, 296-302, A. Droste et al., Biochem. Biophys. Res. Comm., 256, 110-113, 1999).

### EXAMPLE F1

In this experiment the capability of the tested substances to displace ³H-dexamethasone from the bond sites for the glucorticoids present in human monocytes has been evaluated.

The monocytes have been isolated from human blood by a method based on a density gradient (Ficoll-Hypaque d=1.077). The isolated cells have been transferred in test tubes (1×10⁶ cells, the measurements have been carried out in duplicate) containing the culture medium RPMI 1640 and glutamine 1%. To each test tube, in the order, ³H-dexamethasone (50 nM in DMSO) and the tested compounds dissolved in the same solvent (DMSO) at the concentrations indicated in the Tables reported hereunder have then been added. The test tube content has been mixed using a Vortex equipment. The test tubes have then been incubated at 37° C. for 1 hour.

After incubation, the cells have been washed 4 times with a saline solution in phosphate buffer cooled in ice bath (PBS, 0.01 M) and the amount of [³H]-dexamethasone bound to the cells has been determined by liquid scintigraphy. For each sample the concentration of [³H]-dexamethasone bound to the cells (femtomoles (fmoles)/ml=10⁻¹⁵ moles/ml) has been calculated by substracting from the measured values the non specific bond value, and then multiplying by the ratio molarity/radioactivity.

The experiments have been carried out using the following compounds:
Hydrocortisone-21-(4-nitrooxybutyrate) (Hydr-C₄—ON₂) prepared as described in patent application WO 98/15568;
Hydrocortisone 21-(4′-nitrooxymethyl)benzoate (Hydr-Ar—ONO₂) (Ex. 2);
Dexamethasone-21-(4-nitrooxybutyrate) (Dex-C₄—ONO₂), sinthesized as described in patent application WO 98/15568;
Dexamethasone 21-(4′-nitrooxymethyl)benzoate (Dex-Ar—ONO₂) (Ex. 3)
Prednisolone-21-(4-nitrooxybutyrate) (Predn-C₄—ONO₂), sinthesized as described in patent application WO 98/15568;
Prednisolone 21-(4′-nitrooxymethyl)benzoate (Predn-Ar—ONO₂) (Ex. 4);
Prednisolone-21-[2-[4-(3-nitrooxypropyl) piperazin-1-yl]acetate]bis hydrochloride (Predn-pyper-ONO₂) (Ex. 9);
Prednisolone-21-[2-(3-nitrooxypropyl)-piperazin-1-yl]acetate bis trifluoromethanacetate (Predn-pyper-C₃—ONO₂) (Ex. 13).

The data reported in the Tables are expressed in fmole [³H]-dexamethasone/ml.

The results obtained with Hydr-Ar—ONO₂ and for comparison those with Hydr-C₄—ONO₂ are reported in Table 1;
The results obtained with Dex-Ar—ONO₂ and for comparison those with Dex-C₄—ONO₂ are reported in Table 2.

The results obtained with Predn-Ar—ONO₂, Predn-pyper-ONO₂, Predn-pyper-Ar—ONO₂ and for comparison those with Predn-C₄—ONO₂ are reported in Table 3.

The results show that the steroidal nitrooxy derivatives of the invention are more active than those wherein the nitro-oxy group is bound to the C₄ aliphatic bivalent linking group.

### EXAMPLE F2

Influence of the invention compounds on the cardiocirculatory parameters Sprague Dawley normotensive male rats have been divided in groups and treated, respectively, with Prednisolone 21-[(4′-nitrooxymethyl) benzoate](Ex. 3) 5 mg/Kg/die i.p. for 3 weeks and with the corresponding precursor at the same dose. The controls have been treated with the carrier (peanut oil 0.5 ml/rat/die i.p. for 3). At the end of the treatment the average arterial pressure (MABP) and the heart-beat have been controlled in the rats. The basal MABP of the controls has been 110±4 (n=9), in the group treated with prednisolone it has been 154±7 (n=8 p<0.01) and in that treated with the nitrooxyderivative compound 128±7 (n=9 p<0.05). As regards the heart-beat it has been found that the nitrooxy derivative of Prednisolone does not significantly influence said parameter (control 330±32 n=9; treated group 348±18, n=9).

### EXAMPLE F3

### Effect of the Budesonide 21-[(4′-nitrooxymethyl)benzoate](NO-Budesonide) (Ex. 5) vs. the precursor Budesonide on the bronchoconstriction caused by histamine in guinea pigs

Male guinea pigs weighing between 250 and 300 g for several days before the beginning of the experiment have been α-customed to the restrained and whole-body plethysmograph. Histamine (3 mM dissolved in saline solution 0.9%) has been administered by intranasal route for 20 seconds 24 hours before and 15 minutes after the administration of the tested compounds. NO-Budesonide (635 μg/ml) and Budesonide (448 μg/ml) dissolved in a mixture (v/v) DMSO 20%, ethanol 10%, saline physiological solution 70%, or the carrier, have been administered to the animals as aerosols, in a sealed room, using a wright nebulizer operating by compressed air at a pressure of 21.38×10³ Pa (20 p.s.i.) and a flow of 0.5 ml/min. The administration lasted 15 minutes.

To monitor the functionality of the animal airways the “whole body” plethysmography has been used. The animals were watchful and functionality has been determined as specific conductance of the airways (sG_{aw}), expressed in % change of the basal value in the instant immediately after the exposure. To this purpose the animals were provided with a suitable mask and then transferred in a sealed room. The respiratory flow has been determined by a pneumotachograph and a pressure transducer. A decrease in sGaw shows bronchoconstriction.

The data reported in Table 4 show that NO-budesonide completely inhibits (100% inhibition) the bronchoconstriction caused by histamine. Budesonide administered at the same molar dose on the contrary worsens the bronchoconstriction caused by histamine.

### EXAMPLE F4

### Comparison between the anti-arthritic activity of prednisolone-21-(4′-nitrooxymethyl) benzoate (Ex. 4) vs. prednisolone

In this pharmacological experiment in vivo the antiarthritic activity of prednisolone-21-(4′-nitrooxymethyl)-benzoate vs. prednisolone has been determined in a model of arthritis in rats.

Lewis female rats weighing 150-200 g fed by a standard diet and with free access to water have been stabulated with cycles of 12 hours light/dark.

To carry out the experiment, the rats were anaesthetized with halothane (day zero), then at the base of the tail, by intradermal injection, a collagen suspension II/Freund's incomplete adjuvant (400 μg/rat) was injected, prepared as described hereinafter: nasal bovine collagen of type II (Sigma-Aldrich, 4 mg/ml) has been dissolved in acetic acid (0.01 M) and emulsified with a same volume of cold Freund's incomplete adjuvant (Sigma-Aldrich).

The arthritic pathology became evident between the 11^{th} and the 13^{th} day, with a maximum inflammation at the 18^{th} day in untreated rats.

From the 12th to the 18th day subsequent to the injection, the rats, divided in 3 groups of 10 animals each, have been treated i.p. according to the following protocol:
Group 1: prednisolone-21-(4′-nitrooxymethyl)benzoate (4 mmoles/kg);
Group 2: prednisolone (4 mmoles/kg);
Group 3: carrier control (peanut seed oil 0.5 ml/kg, i.p.).

A fourth group of healthy rats (naive) has been taken as a further reference.

During the treatment with the tested compounds the anti-arthritic activity has ben evaluated by the following parameters:
average paw volume determined by a plethysmometer;
clinical evaluation of the hip functionality by an arbitrary score from 0 (absence of inflammation) to 3 (serious inflammation, which affects both the hip articulation and the animal paw).

On the 18th day the rats were sacrificed and the diameters of the femoral articulations of the animal hind legs were determined after skin removal; and an histological analysis of the articulations was carried out.

From the histological tissue analysis it resulted that in the group treated with prednisolone-21-(4′-nitrooxymethyl)benzoate normal both the synovial inflammation and the infiltration of inflammatory cells in cartilages were minimal, without compromizing the cartilages, while in the group of rats treated with prednisolone cartilage ulcerations and synovial inflammation were present, although at a lower extent compared with the untreated control group.

In table 5 there are reported:
the articulation sizes expressed in mm,
the percent reduction of the articulation size calculated with respect to the control rat group treated only with the collagen suspension,
the paw average volume, expressed in ml, daily determined,
the score evaluation of the hip functionality.

The results show that Prednisolone-21-(4′-nitrooxy methyl)benzoate has a strong antiinflammatory activity in the arthritis caused by collagen in rats, that is higher than that of Prednisolone on the considered parameters.

### EXAMPLE F5

Osteoclastic activity of Prednisolone-21-(4′-nitrooxymethyl) benzoate (Ex. 4) vs. Prednisolone

Administration of Prednisolone and generally of glucocorticoids causes an increase of the bony metabolism with consequent bony weight loss which causes a high risk of osteoporosis development and consequent bony fragility.

A suspension of primary rat osteoclastes prepared as described in Mancini L. et al., Biochem. Biophys. Res. Comm. 1998, 243, 785-790, has been placed on two culture plaques having 24 wells, coated with calcium phosphate (apatite). After 30 minutes at 37° C. the non-adhered cells have been removed. To each plaque prednisolone-21-(4′-nitrooxymethyl)benzoate and prednisolone (final concentration 1 nM), respectively, have been added. The plaques have been incubated at 37° C. for 18 hours. Lastly the plaques have been treated with a sodium hypochlorite solution (10% v/v) to remove the cells and determine the areas.

The obtained samples have been analyzed with an inverse microscope (Diaphot TMD; Nikon, Japan) connected to an imagine acquisition system (Argus-10, Hamamatsu Photonics, Enfield, UK). For each plaque the sum of the single reabsorption areas has been calculated and the obtained values have been expressed in percentage with respect to the value of the well average area.

The results are reported in Table 6 and show that while prednisolone stimulates the osteoclast activity the prednisolone-21-(4′-nitrooxymethyl)benzoate does not cause bony reabsorption.

### EXAMPLE F6

Determination of the gastric damage caused by ischaemia-reperfusion of Prednisolone-21-(4′-nitrooxymethyl) benzoate vs. Prednisolone

In this experiment the effects of prednisolone-21-(4′-nitrooxymethyl)benzoate and prednisolone on the gastric damage caused by ischaemia-reperfusion have been compared.

The celiac arteries of anaesthetized rats (6 animals/group) have been temporarily occluded with surgical forceps and a HCl solution (1 ml, 0.1 N) has been introduced in the gastric lumen. After 30 minutes from the introduction of the acid solution the circulation has been reactivated and after 60 minutes from the restarting of the blood circulation the gastric damage has been determined by a lesion intensity index score (LI).

Prednisolone-21-(4′-nitrooxymethyl)benzoate and prednisolone (28 mmoles/kg) have been administered to rats by os 2 hours before the ischaemia.

The results reported in Table 7 show that the prednisolone increases the gastric damage caused by ischaemia-reperfusion, while Prednisolone-21-(4′-nitrooxy-methyl) benzoate does not worsen the experimentally caused gastric ulcer.

### EXAMPLE F7

### Effect of Prednisolone-21-(4′-nitrooxymethyl)benzoate and Prednisolone on the recovery of the motor functions in rats after induction of spinal lesions from trauma

Rats (no. 3 groups of 10 animals each) have been subjected to a trauma of the spinal cord at the thoracic level by a weight fall (10 g). In this way a spinal lesion is provoked, which determines a remarkable compromising condition of the motor function. After the trauma, rats are treated once a day for 5 days with Prednislone-21-(4′-nitrooxymethyl)benzoate (dissolved in saline solution/ethanol 1:8, 20 mg/kg, s.c.) and Prednisolone (20 mg/kg, s.c., likewise dissolved) or with the only carrier.

The animal behaviour is evaluated on the third, fifth and seventh day subsequent to the trauma by a multiple score (BBB score). In the used score the zero value is assigned when the condition of the motor function is severely compromised (the animal does not walk); the 20 value corresponds to the normal motor functionality.

The results reported in Table 8 show that the treatment with prednisolone-21-(4′-nitrooxymethyl)benzoate, differently from the comparative corticosteroid, induces a lesion recovery.

TABLE 1
Receptor binding (GR binding Assay): affinity of the tested compound
for the receptor site, expressed in fmoles bound ³H Dexamethasone/ml
of the nixtrooxyderivatives of hydrocortisone

| | | Bound ³H Dexamethasone |
|---|---|---|
| Compound | Dose (μM) | (fmoles/ml) |
| Hydr-C₄—ONO₂ (comp.) | 10 | 4.3 |
| Hydr-C₄—ONO₂ (comp.) | 0.3 | 25.6 |
| Hydr-Ar—ONO₂ | 10 | 0 |
| Hydr-Ar—ONO₂ | 0.3 | 17.3 |

TABLE 2
Receptor binding (GR binding Assay): affinity of the tested compound
for the receptor site, expressed in fmoles bound ³H Dexamethasone/ml,
of the nitrooxyderivatives of Dexamethasone

| | | Bound ³H Dexamethasone |
|---|---|---|
| Compound | Dose (μM) | (fmole/ml) |
| Dex-C₄—ONO₂ (comp.) | 0.1 | 2.6 |
| Dex-Ar—ONO₂ | 0.1 | 1.1 |

TABLE 3
Receptor binding (GR binding Assay): affinity of the tested compound
for the receptor site, expressed in fmoles bound ³H Dexamethasone/ml,
of Prednisolone and corresponding derivatives

| | | Bound ³H Dexamethasone |
|---|---|---|
| Compound | Dose (μM) | (fmole/ml) |
| Predn-C₄—ONO₂ (comp.) | 1 | 9.9 |
| Predn-Ar—ONO₂ | 1 | 0 |
| Predn-pyper-ONO₂ | 1 | 5.5 |
| Predn-pyper-C₃—ONO₂ | 1 | 2.4 |

TABLE 4
Example F3: variation between the values of the specific conductance
of the airways (sG_{aw}) measured at t0 = 24 hours before and
t1 = 15 minutes after the inhalation of the tested compounds,
in animals (guinea pigs) treated respectively with carrier, budesonide-NO
or budesonide at equimolar doses.

| | Carrier | Budesonide-NO | Budesonide (comp.) |
|---|---|---|---|
| Dose | — | 635 | 448 |
| (μg/ml) | | | |
| t0 | −25.9 ± 10.4 | −29.7 ± 9.8 | −28.7 ± 8.8 |
| t1 | −15.4 ± 7.4 | +1.4 ± 7.9 | −42.6 ± 15.9 |

TABLE 5

| | | Hind Legs | | Leg | Hip |
|---|---|---|---|---|---|
| | Dose | Articulations | | Average | Functionality |
| | (μmoles/ | Sizes | Reduction | Volume | Evaluation |
| Treatment | kg) | (mm) | % | (ml) | (Score) |
| Naive | — | 5.9 | — | 1.1 | 0 |
| Control | — | 7.03 | 0 | 1.5 | 1.6 |
| Predn-Ar—ONO₂ | 4 | 5.7 | 18.6 | 1.1 | 0.1 |
| Predn (comp.) | 4 | 6.1 | 12.7 | 1.26 | 0.6 |

TABLE 6
Example F5: effect of Prednisolone and
Prednisolone-21-(4′-nitrooxymethyl)benzoate
(Predn-Ar—ONO₂) on the osteoclastic
activity in vitro

| | | % Reabsorbed Area with |
|---|---|---|
| Compound | Conc. (nM) | Respect to Control Area |
| Control | — | 100 |
| Prednisolone (comp.) | 1 | 148 |
| Pred-Ar—ONO₂ | 1 | 90 |

TABLE 7
Example F6: Worsening of the gastric lesions
(LI) induced in rat due to administration of
Prednisolone and Prednisolone-21-(4′-nitrooxymethyl)
benzoate(Predn-Ar—ONO₂)

| Compound | Dose (μmoles/kg) | Lesion Index (LI) |
|---|---|---|
| Control | — | 3 ± 1 |
| Prednisolone | 28 | 44 ± 2.5 |
| Pred-Ar—ONO₂ | 28 | 7 ± 2.1 |

TABLE 8
Example F7: recovery of the motor function after induced spine
trauma and subsequent treatment with Prednisolone (comparative)
and Prednisolone-21-(4′-nitrooxymethyl) benzoate

| | Motor Behaviour Evaluation | | |
|---|---|---|---|
| | (BBB score) | | |
| Compound | 3^{rd} day | 5^{th} day | 7^{th} day |
| Control | 4 | 6 | 8 |
| Prednisolone (comp.) | 1 | 2 | 3 |
| Pred-Ar—ONO₂ | 8 | 14 | 17 |

1. Nitrooxy derivatives of steroidal compounds of general formula
B—X₁—NO₂ (I)
or esters or salts thereof, wherein:
B is a steroidal radical having the following structure: 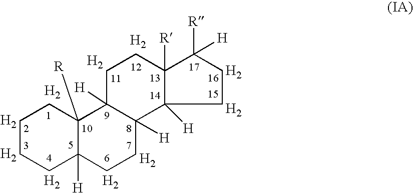
wherein at the place of the hydrogen of the CH group, or of the two hydrogens of the CH₂ group indicated in the general formula, there can be the following substituents:
in position 1-2: a double bond;
in position 2-3 the following substituent: 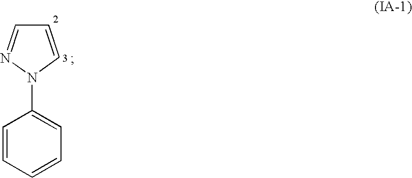
in position 2: Cl, Br;
in position 3: oxo, —O—CH₂—CH₂—Cl, OH, OCH₃;
in position 4-5: a double bond;
in position 5-6: a double bond;
in position 6: Cl, F, Br, CH₃, —CHO;
the ring defined by the carbon atoms numbered 1, 2, 3, 4, 5, and 10 is an aromatic ring when B is the residue of an estrogen;
in position 7: Cl, OH;
in position 9: Cl, F, Br;
in position 11: OH, oxo, Cl;
in position 16: CH₃, OH, ═CH₂;
in position 17: OH, CH₃, OCO(O)ᵤₐ(CH₂)ᵥₐCH₃ wherein ua is an integer equal to 0 or 1, va is an integer from 0 to 4, ethynyl (C≡CH); or 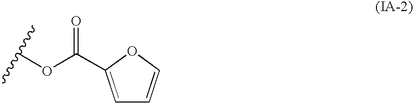
in position 16-17 the following groups: 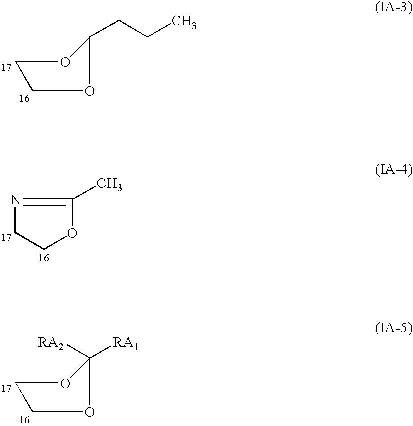
wherein RA₁ is H, CH₃; RA₂ is C₁-C₁₀ linear o branched alkyl chain, preferably C₁-C₃, still more preferably CH₃, or a saturated cycloaliphatic ring having 5-6 carbon atoms or an aromatic ring optionally substituted in para position with N(R_{1c})₂ wherein R_{1c} is a C₁-C₁₀, preferably C₁-C₄, linear or branched alkyl; R and R′, equal to or different from each other, can be hydrogen or C₁-C₄ linear or branched alkyls, preferably R═R′═CH₃ or R═R′═H;
preferably B is a corticosteroid residue;
R″ in position 17 is a bivalent radical having one of the following meanings:
IB) —(CO-L)ₜ-(X_{O})ₜ₁-, wherein t=1 and t1 is 0 or 1, preferably 0;
IC) -L-(X₀)ₜ₁-, wherein t1 is 0 or 1, preferably 1;
the bivalent linking group L has the following meaning:
(CR₄R₅)ₙₐ(O)_{nb}(CR_{4′}R_{5′})_{n′a}(CO)_{n′b}(O)_{n″b}(CO)_{n′″b}(CR_{4′}R_{5′})_{n″a}
wherein na, n′a, and n″a, equal to or different from each other, are integers from 0 to 6, preferably 1-3; nb, n′b, n″b and n′″b, equal to or different from each other, are integers equal to 0 or 1; R₄, R₅, R_{4′}, R_{5′}, R_{4″}, R_{5″}, equal to or different from each other, are selected from H, C₁-C₅, preferably C₁-C₃, linear or branched alkyl;
X_{O}≡O—, —NH—, —NR_{1c}— wherein R_{1c} is as above defined;
the bond between the steroid and the linking group X₁ is of ester or amidic type;
X₁ is a bivalent linking group selected from the following:
Y_{AR1}: 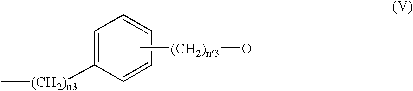
wherein n3 is an integer from 0 to 5 and n3′ is an integer from 1 to 3;
Y_{AR2}: 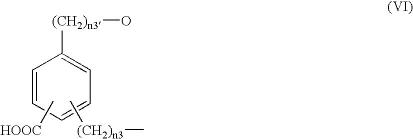
wherein n3 and n3′ have the above meaning. Yₚ: 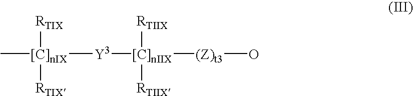
wherein:
nIX is an integer from 0 to 10, preferably 1-3;
nIIX is an integer from 1 to 10, preferably 1-5;
R_{TIX}, R_{TIX′}, R_{TIIX}, R_{TIIX′}, equal to or different from each other are H or C₁-C₄ linear or branched alkyl; preferably R_{TIX}, R_{TIX′}, R_{TIIX}, R_{TIIX′} are H; Y³ is a saturated, unsaturated or aromatic heterocyclic ring, having 5 or 6 atoms, containing from one to three heteroatoms, preferably from one to two, said heteroatoms being equal or different and selected from nitrogen, oxygen, sulphur, preferably nitrogen;
t3 is zero or 1;
Z has the following meaning: 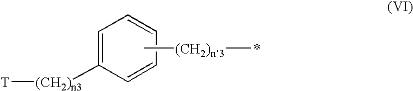
wherein:
shows the position of the ONO₂ group;
T has the following meanings:
—COX₃—, —X₃CO—, wherein X₃═S or Xₒ as above defined;
—X₃— as above defined;
n3 and n′3 are as above defined.

2. Compounds according to claim 1, wherein Y³ is selected from the following bivalent radicals:

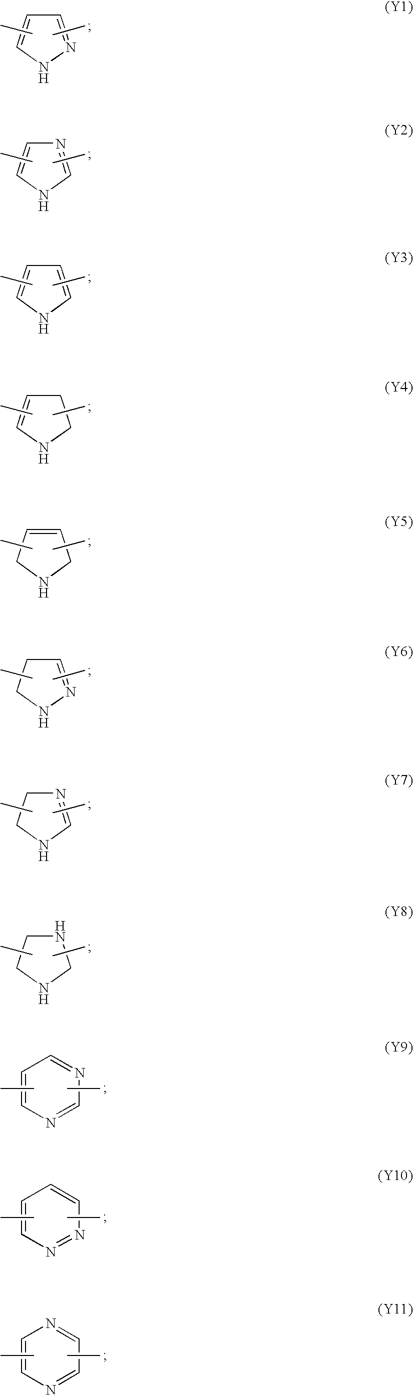

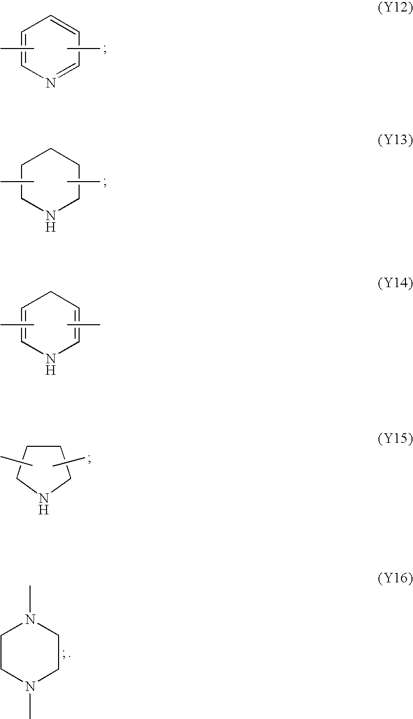

3. Compounds according to claim 2, wherein Y³ is selected from the following: (Y12), having the two free valences in the ortho positions with respect to the nitrogen atom; (Y16) with the two valences linked to the two heteroatoms, (Y1) (pyrazol) 3,5-disubstituted; (Y16) is particularly preferred.

4. Compounds according to claim 1, wherein the precursor of B is selected from the following: Budesonide, Hy-drocortisone, Alclomethasone, Algestone, Beclomethasone, Betamethasone, Chloroprednisone, Ciclesonide, Clobetasol, Clobetasone, Clocortolone, Cloprednol, Cortisone, Corticosterone, Deflazacort, Desonide, Desoximethasone, Dexa-methasone, Dexamethasone 17-furoate, Diflorasone Diflu-cortolone, Difluprednate, Fluazacort, Flucloronide, Flumethasone, Flunisolide, Fluocinolone Acetonide, Fluocinonide, Fluocortyn Butyl, Fluocortolone, Fluorometholone, Fluperolone Acetate, Fluprednidene Acetate, Fluprednisolone, Flurandrenolide, Formocortal, Halcinonide, Halobetasol Propionate, Halomethasone, Halopredone Ace-tate, Hydrocortamate, Itrocinonide, Loteprednol Eta-bonate, Meclonisone, Medrysone, Meprednisone, Methylprednisolone, Momethasone Furoate, Paramethasone, Prednicarbate, Prednisolone, Prednisolone 25-Diethylaminoacetate, Prednisolone Sodium Phosphate, Prednisone, Prednival, Prednylidene, Rofleponide Rimexolone, Triamcinolone, 21-Acetoxypregnenolone, Cortivazol, Amcinonide, Fluticasone Propionate, Mazipredone, Taucorten, Tixocortol, Triamcinolone Hexacetonide; Ursodesoxycholic acid, Chenodesoxycholic acid; Mytatrienediol, Moxestrol, Ethynyl-estradiol, Estradiol, Mestranol; methyl (20R)-6-alpha, 9alpha-difluoro-11beta-hydroxy-16alpha, 17alpha propyl methylene dioxyandrosta-1,4-dien-3-one-17beta-carboxylate.

5. Compounds according to claim 1, wherein when B is the residue of a corticosteroid, R″=IB with t=1 and t1=0; preferably in the formula of the linking group L na=nb=n′b=1; n′a=n″b=n′″b=n″a=0; R₄=R₅=H; or n′b=n″b=1 and all the other na, nb, n′a, n′″b, n″a are equal to zero.

6. Compounds according to claim 1, wherein when B is the residue of a biliary acid, R″=IC with t1=1; preferably in the formula of the linking group L na=n′b=1, n′a=2, n″b=n′″b=n″a=nb=0, R₄=CH₃, R₅=R_{4″}=R_{5″}=H.

7. Compounds according to claim 1, wherein when B is the residue of an estrogen
R″=IC wherein t1=0 and in the formula of the linking group L nb=n′b=1; na=n′a=n″b=n′″b=n″a=0, the other substituent of the carbon atom in position 17 is preferably H or ethynyl, and in position 3 one of the substituents is optionally the —R″—X₁—NO₂ group (R″ as above defined); or
in position 3 the —R″—X₁—NO₂ group is present, R″ being as above when B is the residue of an estrogen, then the substituents of the carbon atom in position 17 in formula (IA) of B are the following:
R″=—O—, wherein the oxygen free valence is saturated with H;
H is substituted with a group different from OH.

8. Compounds according to claim 1, selected from the following:
Hydrocortisone 21-(4′-nitrooxymethyl) benzoate 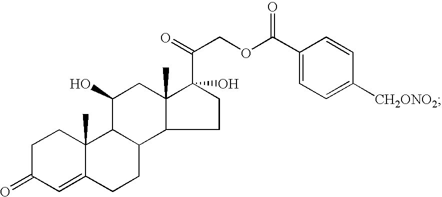
Hydrocortisone-21-[2-[6-(nitrooxymethyl)-2-pyridinyl]acetate] 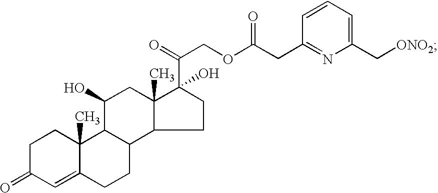
Hydrocortisone-21 [2-[4-[2-[4-(nitrooxymethyl)benzoyloxy]ethyl]piperazinyl]acetate] 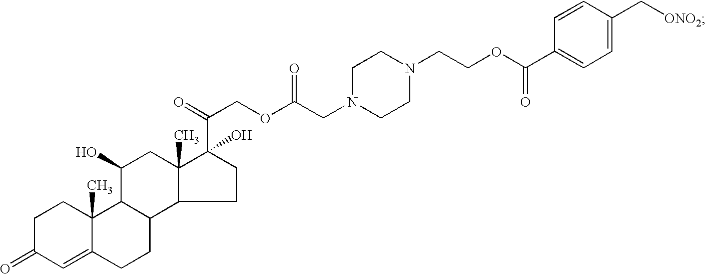
Hydrocortisone-21-[2-[4-[3-(nitrooxy)propyl]-1-pipera-zinyl]acetate] 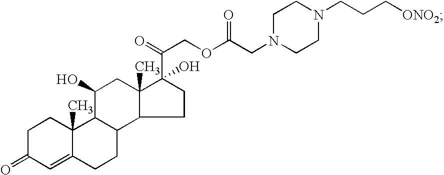
Dexamethasone 21-(4′-nitrooxymethyl)benzoate 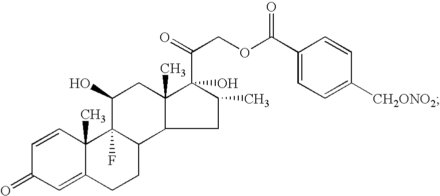
Dexamethasone-21-[2-[4-[3-(nitrooxy)propyl]-1-piperazi-nyl]acetate] 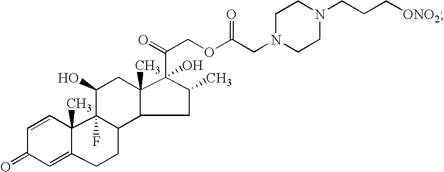
Dexamethasone-21 [2-[4-[2-[4-(nitrooxymethyl)benzoyloxy]ethyl]-1-piperazinyl]acetate] 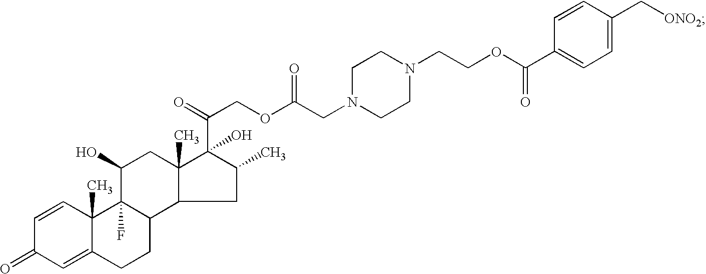
Dexamethasone-21-[2-[6-(nitrooxymethyl)-2-pyridinyl]acetate] 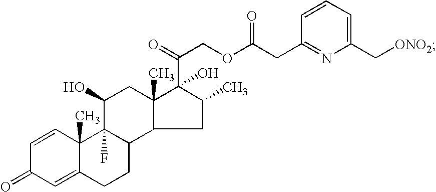
Prednisolone 21-(4′-nitrooxymethyl)benzoate 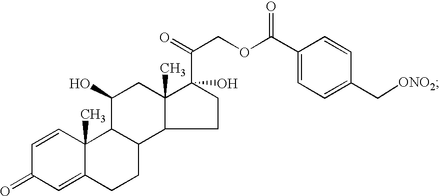
Budesonide 21-(4′-nitrooxymethyl)benzoate: 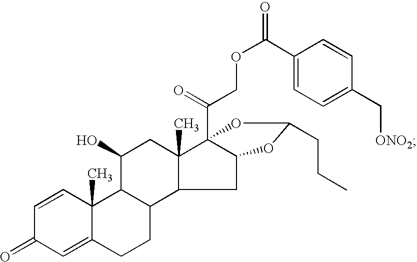
Budesonide-21-[2-[6-(nitrooxymethyl)-2-pyridinyl]acetate] 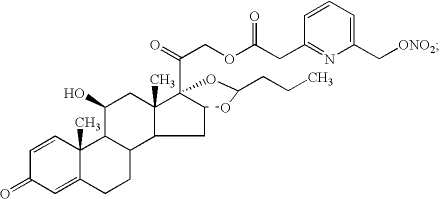
Budesonide-21-[2-[4-[3-(nitrooxy)propyl]-1-piperazinyl]acetate] 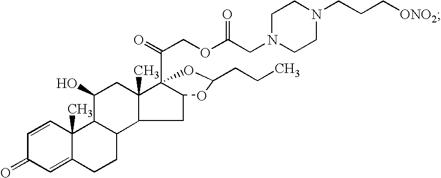
Budesonide-21 [2-[4-[2-[4-nitrooxymethyl)benzoyl oxy]ethyl]-1-piperazinyl]acetate] 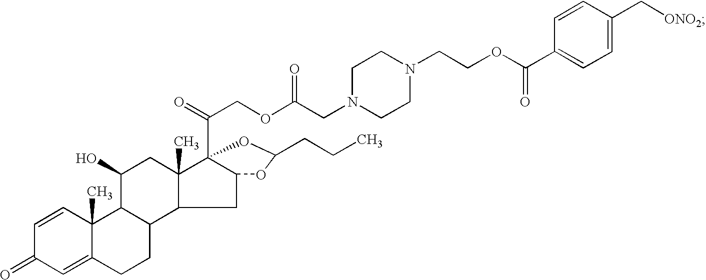
Flumethasone 21-(4′-nitrooxymethyl)benzoate: 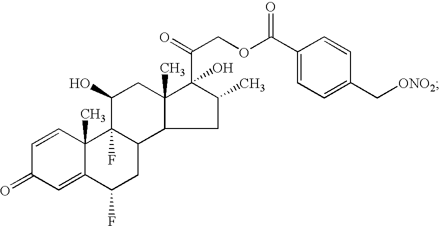
Flumethasone-21-[2-[6-(nitrooxymethyl)-2-pyridinyl]ace-tate] 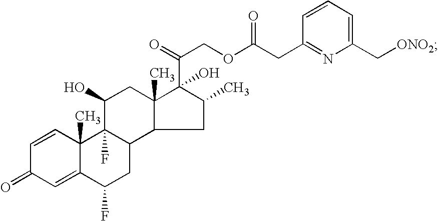
Flumethasone-21-[2-[4-[2-[4-(nitrooxymethyl)benzoyloxy]-ethy]-1-piperazinyl]acetate] 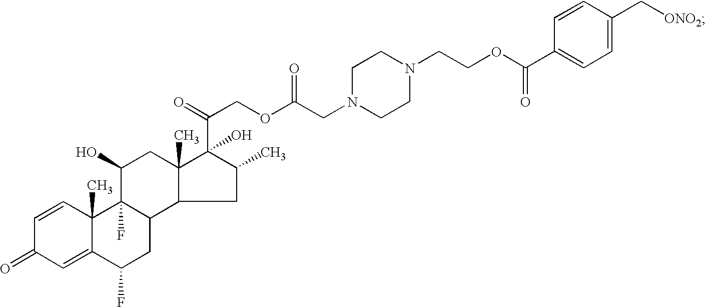
Flumethasone-21-[2-[4-[3-(nitrooxy)propyl]-1-piperazinyl]acetate] 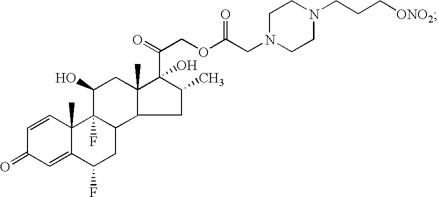
Prednisolone-21-[2-[6-(nitrooxymethyl)-2-pyridinyl]ace-tate] 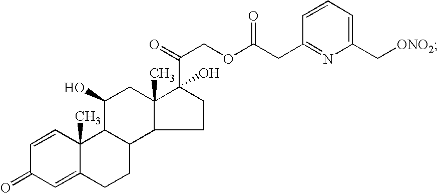
Prednisolone-21-[2-[4-(3-nitrooxy)propyl) piperazin-1-yl]acetate]and the corresponding bishydrochloride salt: 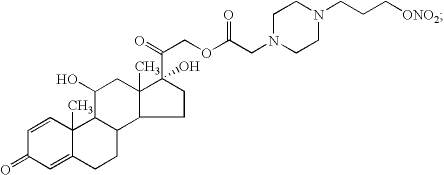
Prednisolone-21-[2-[4-[2-[(4′-nitrooxymethyl)benzoyl oxy]ethyl]piperazin-1-yl]acetate]and the corresponding bishydrochloride salt: 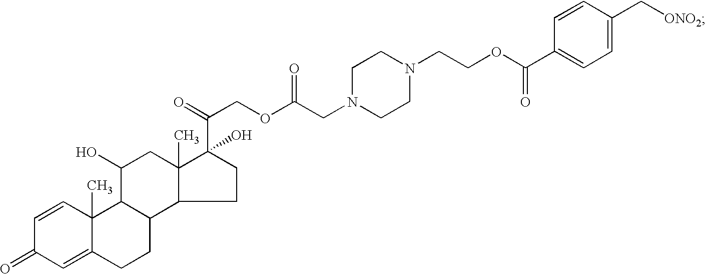
Flunisolide-21-[2-[4-[3-(nitrooxy)propyl]-1-piperazinyl]acetate] 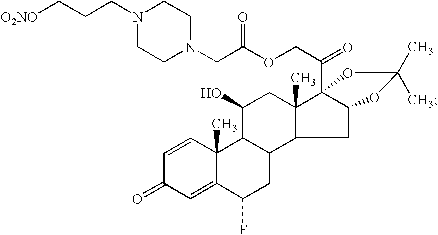
Flunisolide-21-[(4′-nitrooxymethyl)]benzoate 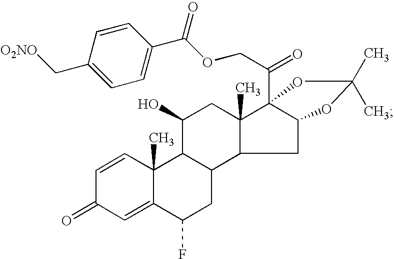
Flunisolide-21-[2-[4-[2-[4-(nitrooxymethyl)benzoyloxy]ethyl]-1-piperazinyl]acetate] 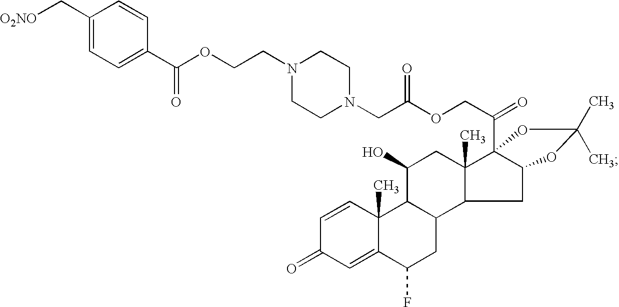
Flunisolide-21-[2-[6-(nitrooxymethyl)-2-pyridinyl]aceta-te] 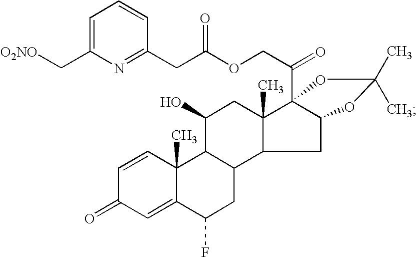
Flunisolide 21-[(4′-nitrooxymethyl)benzoate)]

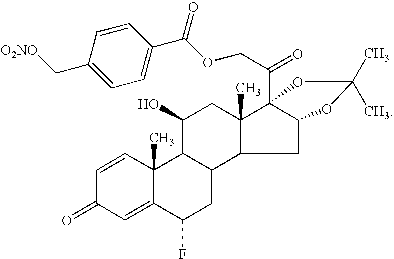

9. Use of the compounds according to claim 1, for the preparation of medicaments.

10. Use of the compounds according to claim 1, as drugs having antiinflammatory activity at peripheral level.

11. Use of the compounds according to claim 1, for the therapy of neurodegenerative diseases on an inflammatory and traumatic basis of the nervous system.

12. Use of the compounds according to claim 1, as drugs having an antiarthritic activity.

13. Use of the compounds of claim 1, as drugs having an immunodepressive activity.

14. Use of the compounds according to claim 1, as drugs having an angiostatic/angiogenetic activity.

15. Use of the compounds according to claim 1, as drugs having an antiasthmatic activity.

16. Use of the compounds according to claim 1, in substitutive hormonal therapies, preferably in the post-menopause therapy.

17. Use of the compounds according to claim 1, in rheumatic disease therapies.

18. Use of the compounds according to claim 1, in renal disease therapies.

19. Use of the compounds according to claim 1, in bronchial disease therapies.

20. Use of the compounds according to claim 1, in ocular disease therapies.

21. Use of the compounds according to claim 1, in dermatologic disease therapies.

22. Use of the compounds according to claim 1, in autoimmune disease therapies.

23. Use of the compounds according to claim 1, in tumoral process therapies, optionally in combination with chemotherapic and/or radiotherapic treatments.

24. Use according to claim 10, in inflammatory pathologies affecting the gastrointestinal system.

25. Use of the compounds according to claim 1, wherein the glucocorticoid compounds are used in respiratory pathologies characterized by broncho-obstructive events.

26. Pharmaceutical formulations comprising the compounds of claim 1.
